(19) United States
(12) Patent Application Publication
(10) Pub. No.: US 2011/0153302 A1
(43) Pub. Date: Jun. 23, 2011

(54) IDENTIFICATION OF DRUG EFFECTS ON SIGNALING PATHWAYS USING INTEGER LINEAR PROGRAMMING

(75) Inventors: Alexander Mitsos, Lexington, MA (US); Leonidas G. Alexopoulos, Glyfada (GR)

(73) Assignee: Massachusetts Institute of Technology, Cambridge, MA (US)

(21) Appl. No.: 12/952,735

(22) Filed: Nov. 23, 2010

Related U.S. Application Data
(60) Provisional application No. 61/264,101, filed on Nov. 24, 2009.

(51) Int. Cl. G06G 7/58; A61K 31/517; A61K 31/5377; A61P 43/00

(52) U.S. Cl. 703/11; 514/266.24; 514/234.5

(57) ABSTRACT

Methods and algorithms for modeling biological interaction networks using integer linear programming (ILP) are provided. Methods to identify the effect of a drug on such interaction networks are also provided. Methods to use ILP-base modeling of biological interaction networks and drug effects to personalize clinical interventions are also provided.

### RELATED APPLICATION

This application claims priority under 35 U.S.C. §119(e) to U.S. provisional patent application Ser. No. 61/264,101, filed Nov. 24, 2009, the entire contents of which are incorporated herein by reference.

### BACKGROUND OF THE INVENTION

Understanding the mechanisms of cell function and drug action is a major endeavor in the pharmaceutical industry. Drug effects on cells are governed by the intrinsic properties of the drug, for example, the drug's binding properties, such as its binding selectivity and affinity to a target molecule and its effect on the bound target molecule, and by the specific signal transduction network, or molecular interaction topology, of the target cell.

Cellular signal transduction networks are usually represented as graphical signaling pathway maps. Each cell type has distinct signaling transduction mechanisms, and several diseases arise from alterations on the signaling pathways. Disease states, in turn, can affect the signal transduction network of a cell and, accordingly, modulate or alter the effect a drug has on a cell.

Drugs that target cellular signal transduction pathway members, for example, small-molecule kinase inhibitors, have emerged as novel pharmaceutical interventions that aim to block certain signaling pathways in order to alter a cell state, for example, in order to reverse an abnormal phenotype of a diseased cell. While such drugs are generally designed to bind and inhibit particular target molecules with high specificity, little is known about how they act on an “operative” signaling network.

### SUMMARY OF THE INVENTION

Traditionally, cellular interaction networks, for example signaling pathways, are represented as graphical interaction maps, for example, as protein interaction networks. Interaction networks typically comprise nodes representing signaling pathway members, for example, genes or proteins, and edges representing potential interactions, for example, activation, inhibition, phosphorylation, or dephosphorylation, that link the nodes. Edges can be directional or nondirectional, and can be functionally signed (e.g., inhibitory/activating) or not. Graphical interaction networks are not executable per se, but can be translated into logic-based network models for computational interaction analysis and prediction and/or assessment of perturbations, for example, as induced by a drug.

Biological interactions are inherently difficult to accurately represent in logical models. Most interactions, for example, protein binding or catalytic reactions, are typically non-discrete processes, and a translation of such biological processes into binary logic models in which each species (e.g., a reaction within a signaling network or a phosphorylation state of a protein) is either in an on or off state (1 or 0, respectively), has been viewed to result in an overly simplistic representation of a complex biological system. In contrast, fuzzy logic and multistate or mixed discrete continuous logic models have been developed that can closely approximate biological interactions. However, the increased degree of realism in such models comes at the cost of increased algorithm complexity, for example, in the form of a large number of free parameters that must be estimated. As a result, conventional “realistic” logic models are beyond today's computational capabilities when the signaling pathway network reaches a certain level of complexity and are, accordingly, limited to modeling simple networks consisting of only few nodes and edges.

Some aspects of this invention are based on the surprising discovery that, in contrast to the traditional view, cellular signaling pathway topologies can efficiently be modeled with integer linear programming (ILP) formulations and that such ILP-based signaling pathway models can be used to characterize and/or predict signaling processes with a high degree of accuracy and realism. Traditionally, ILP-based strategies have not been viewed as adequate for modeling complex biological processes, since most biological interactions, including signaling pathway interactions, such as protein-protein binding, inhibition, activation, phosphorylation, etc., are neither linear, nor discrete, but often follow concentration-dependent, non-discrete kinetics, and often involve equilibrium-shifting and circular regulatory loops. Translating non-discrete, non-linear biological interactions as ILP formulations, for example, ILP formulations comprising binary decision variables representing only an on- and an off-state, has traditionally been viewed as yielding only a crude approximation which, in turn, results in a non-realistic and inaccurate model inferior to more complex algorithms that allow for more “realism” by using real decision variables.

Surprisingly, however, the ILP-based modeling approaches provided herein approximate actual data with superior accuracy as compared to conventional modeling strategies, while providing the computational efficiency and scalability of ILP formulations. For example, as described in the Examples section, an ILP formulation as provided herein achieves a more accurate fit of experimental data, with 98 mismatches of 880 data points as compared to 110 for a conventional GA. Further, based on the computational efficiency of the ILP formulation, this superior accuracy is achieved in only a fraction of the time required for methods using conventional algorithms, e.g., the ILP described in the Examples section required 14.3 seconds to identify the optimal signaling pathway topology, whereas the conventional GA required ˜1 hr to identify a slightly less realistic pathway topology. Importantly, ILP-based interaction network modeling approaches as described herein are highly scalable based on their computational efficiency, thus allowing to model complex interaction networks within user-tolerable computing time frames.

Some aspects of this invention are based on the recognition that even the most complex biological interaction networks, and even networks comprising non-linear, non-discrete interactions, can efficiently and accurately be modeled with ILP formulations. Some aspects of this invention relate to the recognition that such complex networks can be broken down into linear reactions amenable to ILP modeling. Some aspects of this invention relate to the recognition that the linear structure of reactions within a network can be exploited for modeling purposes by using ILP-based formulations to generate a network topology, which is computationally efficient without sacrificing accuracy, or “realism,” of the resulting model. Accordingly, some aspects of this invention provide methods for the application of ILP formulations to model complex biological processes, for example, biological interaction networks (e.g., protein interaction networks, cellular signaling pathway topologies, gene expression networks), and the effects of stimuli and drugs on such processes, in an accurate and computationally efficient manner.

Some aspects of this invention are based on the recognition that some biological interaction networks, for example, cellular signaling pathway networks, are cell type and/or cell state specific, and that generic network models, for example, cellular signaling pathway topologies curated from literature or pathway databases, are insufficient to accurately reflect active network structures, for example, signaling pathway connections, in a given cell, cell type, or cell state of interest [18]. Some aspects of this invention provide methods and algorithms to generate cell-specific interaction topologies, for example, cell-specific signaling pathway topologies, based on actual observations made in the respective cell, cell type, or cell state of interest. In some embodiments, a cell specific interaction topology, for example, a cell-specific signaling pathway topology, is generated by optimizing a generic interaction topology, for example, a generic signaling pathway topology curated from literature or a signaling pathway database, based on actual observations relevant to the topology in the cell of interest, for example, of phosphoproteomics data. In some embodiments, the optimization is effected by modifying the generic topology, for example, by deleting non-functional interactions and/or pathway members and/or by adding new functional interactions, and using an ILP formulation to identify an optimal modified topology, or group of topologies, that best fit the actual observations.

Some aspects of this invention are based on the recognition that the effect of a drug on cellular function cannot accurately be characterized and/or predicted solely based on an analysis of the activity of a molecule targeted by the drug, for example, a cellular target protein, but should be assessed, preferably in an unbiased manner, in the context of cellular interaction networks, for example, in the context of cellular signaling pathway topologies. Some aspects of this invention provide methods and algorithms to identify the effect of a drug on a cellular interaction network, for example, on a cell-specific signaling pathway topology, using ILP formulations to integrate actual observations of the drug's effect into logic models of such interaction networks.

Some aspects of this invention are based on the recognition that, in order to correctly predict and/or characterize an effect of a stimulus or drug on a signaling pathway network in a given cell or cell population, it is important that the signaling pathway model employed reflects the actual properties of a specific cell or cell type of interest as accurately as possible. Since signaling pathway networks differ, sometimes significantly, from cell type to cell type and from cell state to cell state, computational prediction and/or analysis of signaling pathway network activities and/or perturbations in a cell population of interest is preferably carried out using a signaling topology specific for the respective cell-type and/or cell-state.

Some aspects of this invention relate to methods for translating biological signaling pathway networks into executable logic models using an integer linear programming strategy, which enables computational analysis and/or prediction of complex signaling pathway interactions in a realistic, yet computationally efficient manner. The methods and models provided herein are useful for the generation and optimization of cell-specific signaling pathway network maps and for the use of such cell-specific maps in computational analysis and characterization and/or prediction of drug effects on cellular signaling and function.

The methods and models provided herein are further useful for the customization of therapeutic interventions based on a cell specific signaling pathway topology generated from a target cell population, for example, a cell population obtained from a tumor in a subject. In some embodiments, methods are provided in which a clinical intervention comprising administration of a drug is selected from a group of candidate interventions based on a cell-specific signaling pathway topology generated from cells targeted by the intervention. In some embodiments, such methods comprise obtaining a target cell population from a subject diagnosed with a disease, wherein the cell population comprises cells in a diseased state, and generating a cell specific signaling pathway topology from the cells. In some embodiments, such methods further comprise detecting abnormal signaling interactions within the cell specific signaling pathway topology, for example signaling interactions that are present in the diseased cells, but not in healthy cells of the same cell type or tissue of origin. In some embodiments, such methods further comprise selecting and/or administering a drug based on the drug targeting a detected abnormal signaling interaction in the diseased cells.

Some aspects of this invention relate to methods for modeling complex cellular signaling pathway networks, or topologies, useful for the analysis of drug effects on particular cell populations. In some embodiments, the term “topology” refers to a graphic or computer-readable and/or—executable representation of an interaction network, for example, of a signaling pathway network. Some aspects of this invention provide methods and algorithms for the optimization of a generic signaling pathway map to a cell specific signaling pathway map based on actual observations made in a cell population of interest. In some embodiments, a method for a generic signaling pathway map optimization is provided that comprises (1) providing a generic signaling pathway topology, for example, a logic-based signaling pathway model; and (2) comparing the generic topology to experimental data obtained from cell population of interest to generate a cell specific signaling pathway topology. In some embodiments, the method further comprises (3) optimizing the cell specific signaling pathway topology to generate an optimized cell specific signaling pathway topology. In some embodiments, the step of comparing and/or the step of optimizing is carried out using a logic-based model algorithm. In some embodiments, and a logic-based model algorithm is an integer linear programming optimization formulation.

Some aspects of this invention address the problem of integrating actual data, for example, of high throughput proteomics data, into a generic pathway topology in order to optimize the generic pathway topology to accurately reflect a signaling pathway network in a specific cell population. In some embodiments, integer linear programming optimization formulations are provided for generic topology optimization and cell specific topology generation.

Some aspects of this invention provide methods and algorithms useful for determining the effect of a drug on a cell-specific signaling topology. In some embodiments, a method for determining the effect of the drug on a cell specific signaling topology is provided that comprises (1) providing a cell specific signaling pathway topology; (2) comparing the cell specific signaling pathway topology in the absence of a drug to the cell specific signaling pathway topology in the presence of a drug. In some embodiments, the cell specific signaling pathway topology provided under (1) is an optimized cell specific signaling pathway topology.

In some embodiments, a method for determining the effect of the drug on a cell specific signaling topology is provided that comprises (1) providing a generic signaling pathway topology, for example, a logic-based signaling pathway model; (2) comparing the generic topology to experimental data obtained from cell population of interest to generate a cell specific signaling pathway topology; and (3) comparing the cell specific signaling pathway topology in the absence of a drug to the cell specific signaling pathway topology in the presence of a drug. In some embodiments, the method further comprises a step of optimizing the cell specific signaling pathway topology to generate an optimized cell specific signaling pathway topology.

Some aspects of this invention relate to the combination of high-throughput signaling network assessments, for example, of phosphoproteomics data, with sophisticated computational techniques to evaluate drug effects on cells. This approach comprises two steps: (1) the generation of a signaling pathway model that simulates cell function and (2) the identification of drug-induced alterations of the modeled pathways. This technology is useful for characterizing on-target as well as off-target effects of candidate drugs on specific cell types and for understanding and monitoring drug effects in normal and diseased cells. The methods disclosed herein can further be used to analyze and/or predict clinical outcome of drug administration, and to improve drug efficacy and safety.

Some aspects of this invention relate to methods of customizing clinical interventions to individual patients based on a cell specific signaling pathway topology generated from a cell or cell population obtained from the patient. In some embodiments, such methods comprise generating a signaling pathway topology that is specific for a cell or a population of cells obtained from the subject. In some embodiments, the cell or cell population obtained from the subject are in a diseased state were derived from a diseased or malignant tissue, for example, malignant cells or cells derived from a tumor. In some embodiments, such methods comprise determining whether the cell specific signaling pathway topology generated from the cells obtained from the subject comprises a biological interaction that can be perturbed by a particular drug to result in a desirable clinical outcome, and, if the signaling pathway topology comprises such an interaction, administering the drug to the subject, or if the signaling pathway topology does not comprise an interaction that can be perturbed by a particular drug to result in a desirable clinical outcome, not administering the drug to the subject.

Some aspects of this invention provide a method of generating a cell-specific interaction network topology using integer linear programming. In some embodiments the interaction network topology is a signaling pathway topology. In some embodiments, the method comprises (a) providing a generic cellular signaling topology comprising a signaling pathway, wherein the signaling pathway comprises a plurality of nodes representing signaling molecules, and wherein the nodes are connected via edges representing reactions within the pathway; (b) determining a quantitative baseline value for a plurality of designated nodes in the generic signaling topology by measuring a quantitative value for each of the plurality of designated nodes in a cell; (c) determining a quantitative value for each of the plurality of designated nodes in the generic signaling topology by measuring a quantitative value for each of the plurality of designated nodes in a cell of the same type as the cell in (b) contacted with a stimulus known to perturb the generic signaling topology; (d) comparing the quantitative baseline value for each of the plurality of designated nodes with the quantitative value for that node determined in the presence of the stimulus known to perturb the generic signaling topology; and (e) generating a cell-specific signaling topology by modifying the generic signaling topology and using an integer linear programming (ILP) algorithm to identify the modified signaling topology that best fits the values determined in (b) and (c) as the cell-specific signaling topology.

In some embodiments, the ILP algorithm defines a pathway as a set of reactions and species, wherein each species is represented by a node in the pathway topology and each reaction is represented by an edge in the pathway topology. In some embodiments, a reaction is defined by three index sets: (1) a set of signaling molecules, (2) a set of perturbations, and (3) a set of reaction products. In some embodiments, the perturbations include inhibitors and activators. In some embodiments, the inhibitors include small-molecule inhibitors, ligand inhibitors, inhibitory drugs, repressors of gene expression, and RNAi agents. In some embodiments, the index sets provided under (1), (2), and (3) are subsets of the species index set. In some embodiments, at least one value in the set of reaction products represents a phosphorylation level of a protein comprised in the set of signaling molecules.

In some embodiments, the ILP algorithm comprises a constraint defining that a reaction takes place only if all reagents and no inhibitors are present. In some embodiments, the ILP algorithm comprises a constraint defining that if a reaction takes place, all products are formed. In some embodiments, the ILP algorithm comprises a constraint excluding reactions without products and/or reactions with neither reagents nor inhibitors. In some embodiments, the ILP algorithm comprises binary decision variables indicating if a reaction is possible or not. In some embodiments, the ILP algorithm comprises integer decision variables. In some embodiments, the ILP algorithm comprises binary decision variables. In some embodiments, at least one of the ILP decision variables is relaxed to continuous. In some embodiments, the relaxation of the at least one of the ILP decision variables does not alter the feasible set. In some embodiments, at least one of the ILP decision variables is a real decision variable. In some embodiments, a reaction that is not possible is represented as an edge not being present, and a reaction that is possible is represented as an edge being present. In some embodiments, step (e) comprises (e.1) determining the fit of the generic model to the observed data; (e.2) modifying the generic signaling topology by adding and/or removing reactions to generate a plurality of modified signal topologies; (e.3) determining the fit of each or of a subset of the plurality of modified signal topologies to the observed data; and (e.4) selecting the modified signal topology exhibiting the best fit to the experimental data as the cell-specific signal topology. In some embodiments, determining the fit of the topology to the experimental data comprises determining the goodness of fit. In some embodiments, determining the goodness of fit comprises calculating the percentage error.

In some embodiments, the modified topology or group of modified topologies with the best fit is/are selected as the cell-specific topology or group of topologies. In some embodiments, the method further comprises a step of (f) optimizing the cell-specific topology to minimize the number of nodes and/or possible reactions without worsening the fit of the topology to the experimental data. In some embodiments, the ILP algorithm is the ILP algorithm as provided in formulation (1), or a reformulation thereof. In some embodiments, the ILP algorithm comprises a constraint as provided in any of formulations (2)-(11), or a reformulation thereof. In some embodiments, the ILP algorithm is the ILP algorithm as provided in formulations (1)-(20), or a reformulation thereof. In some embodiments, the stimulus known to perturb the generic signaling topology comprises an agent activating or inhibiting the activity of a signaling molecule within the signaling topology, or a combination of such agents. In some embodiments, the stimulus comprises a plurality of agents, each agent individually activating or inhibiting the activity of a signaling molecule within the signaling topology.

In some embodiments, the step of (c) comprises a plurality of separate measurements of cells contacted with different stimuli known to perturb the generic signaling topology. In some embodiments, the different stimuli comprise a ligand and/or a small molecule inhibitor of a signaling molecule, or different signaling molecules, comprised in the generic signaling topology, or different combinations of such ligands and/or inhibitors. In some embodiments, the ligands are selected from the group of growth factors, cytokines, intercellular signaling molecules, and intracellular signaling molecules. In some embodiments, the ligands are selected from the group comprising TNF-alpha, IL1-alpha, HGF, INS, and TGF-alpha. In some embodiments, the small molecule inhibitors are selected from the group comprising PI3K, MEK, p38, cMET, and EGFR inhibitors.

In some embodiments, the generic signaling pathway topology comprises at least 10, at least 20, at least 30, at least 40, at least 50, at least 60, at least 70, at least 80, at least 90, at least 100, at least 150, at least 200, at least 250, at least 300, at least 400, at least 500, at least 600, at least 700, at least 800, at least 900, at least 1000, at least 2000, at least 3000, at least 4000, at least 5000, at least 6000, at least 7000, at least 8000, at least 9000, or at least 10000 nodes. In some embodiments, the generic signaling pathway topology represents part of or all of the kinome and/or phosphoproteome of the cell.

Some aspects of this invention provide a method for identifying an effect of a drug on a molecular interaction network in a cell using integer linear programming. In some embodiments, the molecular interaction network is a signaling pathway. In some embodiments, the method comprises (a) providing a cell-specific signaling topology comprising a signaling pathway, wherein the signaling pathway comprises nodes representing signaling molecules, and wherein the nodes are connected via edges representing reactions within the pathway, and wherein the cell-specific signaling topology is based on quantitative values measured in a cell for a plurality of designated nodes comprised in the signaling topology in the absence and the presence of a stimulus known to perturb the signaling pathway, (b) determining drug-induced signaling topology alterations by (b)(i) contacting a cell of the same cell type as the cell in (a) with a drug known or suspected to perturb at least one reaction in the topology provided in (a), in the absence and/or in the presence of the stimulus known to perturb the signaling pathway; (b)(ii) measuring a quantitative value for a plurality of designated nodes in the cell-specific signaling topology in a cell contacted with the drug in the absence and/or the presence of the stimulus known to perturb the cell-specific signaling topology, (b)(iii) generating a cell-specific, drug-induced signaling topology by using an ILP algorithm to adapt the cell-specific signaling topology to best fit the values measured in (b)(ii); and (c) comparing the cell-specific signaling topology provided in (a) to the cell-specific, drug-induced signaling topology generated in (b)(iii), wherein (c)(i) if a reaction is present in the cell-specific signaling topology but absent in the cell-specific, drug-induced signaling topology, the reaction is indicated to be inhibited by the drug; (c)(ii) if a reaction is absent in the cell-specific signaling topology but present in the cell-specific, drug-induced signaling topology, the reaction is indicated to be activated by the drug; and/or (c)(iii) if a reaction is either absent or present in both the cell-specific and the cell-specific, drug-induced signaling topology, the reaction is indicated to not be affected by the drug.

In some embodiments, the ILP algorithm defines a pathway as a set of reactions and species, wherein each species is represented by a node in the pathway topology and each reaction is represented by an edge in the pathway topology. In some embodiments, a reaction is defined by three index sets: (1) a set of signaling molecules, (2) a set of perturbations, and (3) a set of reaction products. In some embodiments, the perturbations include inhibitors and activators. In some embodiments, the inhibitors include small-molecule inhibitors, ligand inhibitors, inhibitory drugs, repressors of gene expression, and RNAi agents. In some embodiments, the index sets provided under (1), (2), and (3) are subsets of the species index set. In some embodiments, at least one value in the set of reaction products represents a phosphorylation level of a protein comprised in the set of signaling molecules.

In some embodiments, the ILP algorithm comprises a constraint defining that a reaction takes place only if all reagents are present. In some embodiments, the ILP algorithm comprises a constraint defining that if a reaction takes place, all products are formed. In some embodiments, the ILP algorithm comprises a constraint excluding reactions without products and/or reactions with neither reagents nor inhibitors. In some embodiments, the ILP algorithm comprises binary decision variables indicating if a reaction is possible or not. In some embodiments, the ILP algorithm comprises integer decision variables. In some embodiments, the ILP algorithm comprises binary decision variables. In some embodiments, at least one of the ILP decision variables is relaxed to continuous. In some embodiments, the relaxation of the at least one of the ILP decision variables does not alter the feasible set. In some embodiments, at least one of the ILP decision variables is a real decision variable. In some embodiments, a reaction that is not possible is represented as an edge not being present, and a reaction that is possible is represented as an edge being present. In some embodiments, step (b)(iii) comprises (b)(iii)(1) determining the fit of the cell-specific signaling topology to the data observed in the presence of the drug; (b)(iii)(2) modifying the cell-specific signaling topology by adding and/or removing reactions to generate a plurality of modified signal topologies; (b)(iii)(3) determining the fit of each of the plurality of modified signal topology to the data observed in the presence of the drug; and (b)(iii)(4) selecting the modified signal topology exhibiting the best fit to the experimental data as the cell-specific, drug-induced signal topology. In some embodiments, determining the fit of the topology to the experimental data comprises determining the goodness of fit. In some embodiments, determining the goodness of fit comprises calculating the percentage error.

In some embodiments, the modified topology with the lowest percentage error is selected as the cell-specific topology. In some embodiments, the ILP algorithm is the ILP algorithm as provided in formulation (1), or a reformulation thereof. In some embodiments, the ILP algorithm comprises a constraint as provided in any of formulations (2)-(4) and/or (6)-(11), or a reformulation thereof. In some embodiments, the ILP algorithm comprises a constraint as provided in formulation (5), or a reformulation thereof. In some embodiments, the ILP algorithm is the ILP algorithm as provided in formulations (1)-(4) and (6)-(11), or a reformulation thereof. In some embodiments, the ILP algorithm is the ILP algorithm as provided in formulations (1)-(11), or a reformulation thereof. In some embodiments, the stimulus known to perturb the generic signaling topology comprises an agent activating or inhibiting the activity of a signaling molecule within the signaling topology. In some embodiments, the stimulus comprises a plurality of agents, each agent individually activating or inhibiting the activity of a signaling molecule within the signaling topology. In some embodiments, the step of (c) comprises a plurality of separate determinations in the presence of different stimuli known to perturb the generic signaling topology. In some embodiments, the different stimuli comprise a ligand and/or a small molecule inhibitor of a signaling molecule, or different signaling molecules comprised in the generic signaling topology, or different combinations of such ligands and/or inhibitors. In some embodiments, the ligands are selected from the group of growth factors, cytokines, intercellular signaling molecules, and intracellular signaling molecules. In some embodiments, the ligands are selected from the group comprising TNF-alpha, IL1-alpha, HGF, INS, and TGF-alpha. In some embodiments, the small molecule inhibitors are selected from the group comprising PI3K, MEK, p38, cMET, and EGFR inhibitors.

In some embodiments, the cell-specific signaling pathway topology comprises at least 10, at least 20, at least 30, at least 40, at least 50, at least 60, at least 70, at least 80, at least 90, at least 100, at least 150, at least 200, at least 250, at least 300, at least 400, at least 500, at least 600, at least 700, at least 800, at least 900, at least 1000, at least 2000, at least 3000, at least 4000, at least 5000, at least 6000, at least 7000, at least 8000, at least 9000, or at least 10000 nodes. In some embodiments, the cell-specific signaling pathway topology is generated based on data representing part or all of the kinome and/or phosphoproteome of the cell.

Some aspects of this invention provide a method for selecting and/or administering a drug to a subject diagnosed with a disease based on an assessment of a biological interaction network in the subject. In some embodiments, the interaction network is a signaling pathway. In some embodiments, the assessment of the biological interaction network is a signaling pathway topology assessment. In some embodiments the drug is a drug targeting a signaling molecule or reaction. In some embodiments, the drug is known to target a signaling molecule or reaction, or other interaction, within a diseased or abnormal cell, cell type, or cell stage within the subject, for example, within a cancer cell of a specific cancer type (e.g., lung cancer, non-small cell lung cancer, breast cancer, colon cancer etc.) or of a specific tissue of origin. In some embodiments, the drug is known to target a molecular interaction within the diseased or abnormal cell, cell type, or cell stage found in the subject based on an assessment of the drug's effects in cells derived from the subject, or based on an assessment of cells representative of the cells found in the subject according to ILP-based interaction network modeling methods as provided herein. In some embodiments, the drug is a drug targeting a molecule or reaction within the interaction network, for example, a signaling molecule within the signaling pathway topology. In some embodiments, the drug is administered to the subject for treatment of the subject, for example, in order to achieve a desired clinical outcome in the subject. In some embodiments, a desired clinical outcome is an inhibition of or a decrease in an aberrantly active or aberrantly increased signaling activity or molecular interaction and/or an activation of or an increase in an aberrantly inactive or aberrantly decreased signaling activity or molecular interaction. In some embodiments, a desired clinical outcome is the prevention, inhibition, delay, amelioration, or reversion of clinical symptoms, for example, of symptoms associated with a disease the subject is diagnosed with, or the prevention, slowing, or retardation, of a disease to progress, for example, to an advanced stage.

In some embodiments, the method comprises (a) generating or obtaining a cell-specific signaling topology from a cell population obtained from a subject, wherein the cell population comprises cells in a diseased state and wherein the signaling topology is generated by using an integer linear programming optimization scheme; (b) comparing the cell-specific signaling topology from the subject to a reference signaling topology; wherein (b)(i) if a signaling molecule and/or reaction is absent in the reference topology and present in the topology of the subject, then treatment of the subject with a drug that inhibits the signaling molecule and/or reaction is indicated; or (b)(ii) if a signaling molecule and/or reaction is present in the reference topology and absent in the topology of the subject, then treatment of the subject with a drug that activates the signaling molecule and/or reaction is indicated. In some embodiments, the method further comprises (c) administering the drug indicated under (b)(i) or (b)(ii) to the subject in an amount sufficient to inhibit or activate the signaling molecule and/or reaction.

In some embodiments, the cell population is derived from a diseased tissue of the subject. In some embodiments, the diseased tissue is malignant tissue. In some embodiments, the drug is chosen from a group of drugs. In some embodiments, the group of drugs comprises small molecule compounds and/or ligands targeting a common signaling molecule and/or reaction implicated in the disease. In some embodiments, the drugs further affect different additional signaling pathway molecules and/or reactions. In some embodiments, the group of drugs comprises EGFR inhibitors. In some embodiments, the group of EGFR inhibitors comprises Lapatinib, Gefitinib, and Erlotinib. In some embodiments, the reference topology is derived or representative of non-diseased cells of the same cell type or tissue of origin as the cells obtained from the subject.

Some aspects of this invention provide a method for selecting and/or administering a drug targeting a signaling molecule or reaction for treatment of a subject diagnosed with a disease based on a signaling pathway topology assessment, the method comprising (a) generating or obtaining a cell-specific signaling topology from a cell population obtained from a subject, wherein the cell population comprises cells in a diseased state; (b) determining whether a reaction or signaling molecule known to be targeted by a particular drug is present or absent in the cell-specific signaling topology, and (c) if the reaction or signaling molecule known to be targeted by the particular drug is absent, administering the drug to the subject in an amount sufficient to modulate the reaction or signaling molecule to achieve a desired clinical outcome, or if the reaction or signaling molecule known to be targeted by the particular drug is absent, not administering the drug to the subject.

In some embodiments, the cell population is derived from a diseased tissue of the subject. In some embodiments, the diseased tissue is malignant tissue. In some embodiments, the drug is chosen from a group of drugs. In some embodiments, the group of drugs comprises small molecule inhibitors targeting a common signaling molecule and/or reaction implicated in the disease. In some embodiments, the drugs further affect different additional signaling pathway molecules and/or reactions. In some embodiments, the group of drugs comprises EGFR inhibitors. In some embodiments, the group of EGFR inhibitors comprises Lapatinib, Gefitinib, and Erlotinib.

The subject matter of this application may involve, in some cases, interrelated products, alternative solutions to a particular problem, and/or a plurality of different uses of a single system or article.

Other advantages, features, and uses of the invention will be apparent from the detailed description of certain non-limiting embodiments, the drawings, which are schematic and not intended to be drawn to scale, and the claims.

### BRIEF DESCRIPTION OF THE DRAWINGS

FIG. 1. Experimental and computational workflow to assess drug effects. (A) A Boolean generic map is assembled from pathway databases and includes stimuli (squares), key measured phosphoproteins (dark circles), and the neighboring proteins (light circles). (B) Cells are treated with a combination of cytokines and selective inhibitors (circles having a thick border) of known effects and an ILP formulation is used to fit the data to the Boolean pathway. (C) A cell-type specific pathway is constructed. (D) Cells are treated with a combination of cytokines and drugs—their effects are assumed unknown—and ILP is used for the second time to fit the drug-induced phosphorylation data. (E) Alterations of the cell-type specific topology reveals drug effects (thick arrows).

FIG. 2. Cell type specific topology using Integer Linear Programming. The ILP algorithm is using a subset of postulated reactions denoted with black and gray arrows in a generic pathway to construct a HepG2 pathway map (black arrows in pathway diagram). Gray triangles show phosphoprotein activation level upon stimuli (columns in top and bottom panels) and inhibitors (subcolumns in top and bottom panels). Shaded background denotes an error between experimental and pathway-inferred responses. Generic topology can hardly represent the HepG2 signaling responses (shaded background in top panel) and pathway optimization is critical to obtain a pathway topology that captures HepG2 function (limited shaded background in bottom panel). Pathways are visualized using Cytoscape [54].

FIG. 3. Drug-induced pathway alterations. (A-D) Arrows denote drug effects, i.e., reactions that are removed from the HepG2 topology by the ILP algorithm in order to fit the drug-altered phosphoprotein dataset. (E-H) Raw data that correspond to drug effects. Lines indicates the signal between 0 minutes (untreated) and “early response” (average signal of 5 and 25 minutes post stimuli). (I) Off-target effect of Gefitinib. Dose response curve shows that the EGFR inhibitor reduces cJUN activation upon IL1a treatment. R² corresponds to linear fit.

FIG. 4. Raw data for the construction of the cell-type specific map and the evaluation of the drug effects. The signals in the Y-axis correspond to the measurements of the phosphorylated residues listed in Materials and Methods. Each column corresponds to cytokine or cytokine mix and each sub-column to the presence of an inhibitor or drug. The numbers to the left are the maximum values across all treatments measured as arbitrary fluorescent intensities.

FIG. 5. Model Validation. The first panel shows the optimization results when the full dataset (shown in FIG. 2) has been used as training dataset. To validate the model, three subsets were created, in which 20% of experimental cases were removed that corresponded to the treatments with PI3K inhibitor (2nd panel), MEK inhibitor (3rd panel), and p38 inhibitor (bottom panel), and the model was trained against them. The data left out was then used as a test dataset for prediction (see highlighted strips in each panel). The error of prediction of the test subsets (error=goodness of fit as describes in Materials and Methods) is shown on the right of each panel.

FIG. 6. Comparison between genetic algorithm and ILP. Both algorithms performed well and achieved very similar solutions. Shaded background denotes inconsistency between predicted values and experimental data: ILP matched all but 98 out of 880 experimental data, as opposed to 110 mismatches in the topology furnished by the GA. The computational time for ILP was 14.3 sec as opposed to approximately one hour for GA.

### DETAILED DESCRIPTION OF CERTAIN EMBODIMENTS OF THE INVENTION

### Introduction

Target-based drug discovery is a predominant focus of the pharmaceutical industry. The primary objective is to selectively target protein(s) within diseased cells in order to ameliorate an undesired phenotype, e.g., unrestrained cell proliferation or inflammatory cytokine release. Ideally, other pathways within the diseased cells, as well as similar phenotypes in other cell types, should remain unaffected by the therapeutic approach. However, despite the plethora of new potential targets that have emerged from the sequencing of the human genome, rather few have proven effective in the clinic [1]. A major limitation is the inability to understand the mechanisms or drug actions either due to the complex signaling transduction networks of cells or due to the complicated profile of drug potency and selectivity.

Finding a drug's target(s) is traditionally based on high-throughput in vitro assays using recombinant enzymes or protein fragments [2]. The main goal is to characterize the drug's biochemical activity (binding affinities that describe potency and selectivity) and depict them in drug-interaction maps [3]. In most cases, once a target is identified, the in vivo effect on the signaling pathway is validated by measuring the drug's efficiency to inhibit the activity, for example, measured as phosphorylation level [4] of a downstream protein. However, beyond that measurement, little is know on how the rest of the signaling network is affected. In addition, in vivo drug effects can hardly be calculated from in vitro assays for several reasons: most drugs, for example, kinase inhibitors, are promiscuous [5], there is discrepancy between in vivo and in vitro binding affinities of drugs [6], and there is an additional discrepancy between in vivo binding affinities and in vivo activity, for example, kinase inhibitor activity, as measured by assessing phosphorylation of a downstream protein.

To address drug effects in more physiological conditions, novel genomic and proteomic tools have recently been developed [7]. In the genomic arena, large-scale mRNA analysis (e.g., [8],[9]) enhanced by computational approaches for drug target deconvolution (e.g., [10],[11]) have been developed. Despite the holistic advantages that genomic approaches have to offer, proteomic-based discovery is a step closer to the function of the cell. Towards this goal, affinity chromatography offers a viable strategy for in-vivo target identification. This approach utilizes a solid support linked to a bait (for example, a candidate drug) to enrich for cellular binding proteins that can subsequently be identified by mass spectrometry (MS) [12]. However, such experiments usually require large amounts of protein, are biased towards more abundant proteins, and frequently yield false positive hits due to nonspecific interactions [13],[14].

In order to circumvent the nonspecific interaction problem, another bait-based strategy uses quantitative MS with “dirty” inhibitors for baits to immobilize the kinome [15],[16]. While this approach significantly reduces the nonspecific interaction problem, it also limits the target-searching space to those kinases with the highest affinity to the bait. More recently, the development of quantitative MS-based proteomics using SILAC technology [14] extended the search space to all targets that do not bind covalently to the drug. However, incorporation of the SILAC's isotopes requires 5 population doublings and, thus, excludes the application on primary cells with limited replication capabilities. Taken together, the conventional techniques listed above can list the binding affinities of all targets to a particular drug but do not provide information as to whether this binding affinity can or will result in inhibiting the transmission of a signal to a downstream protein or how those preferential bindings can collectively affect the signaling network of the cell.

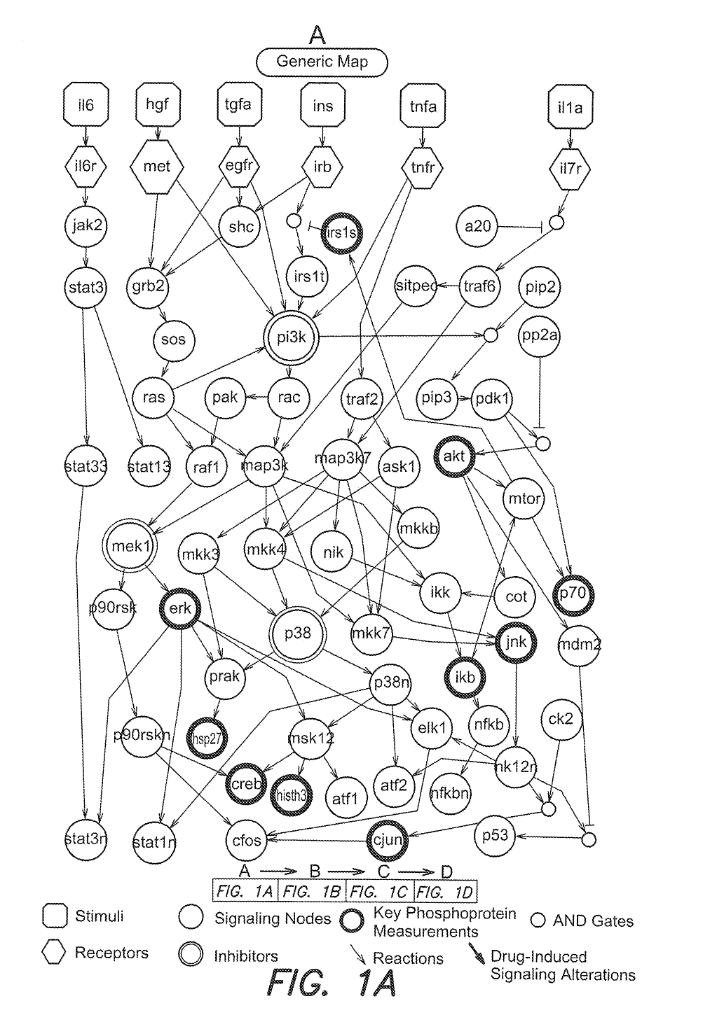

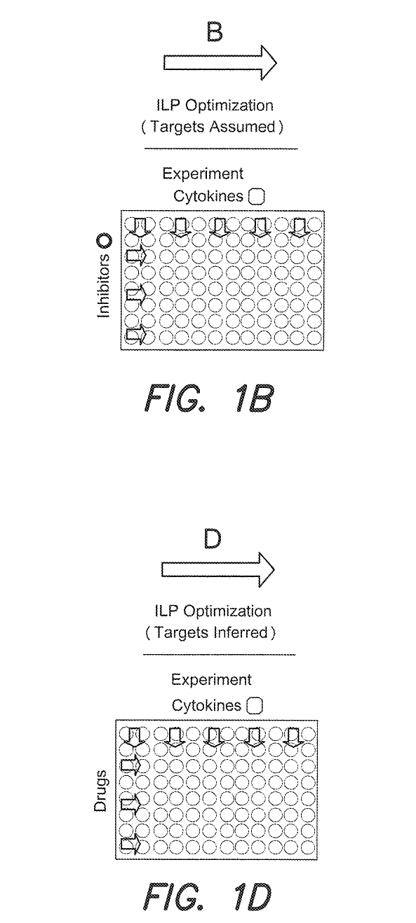

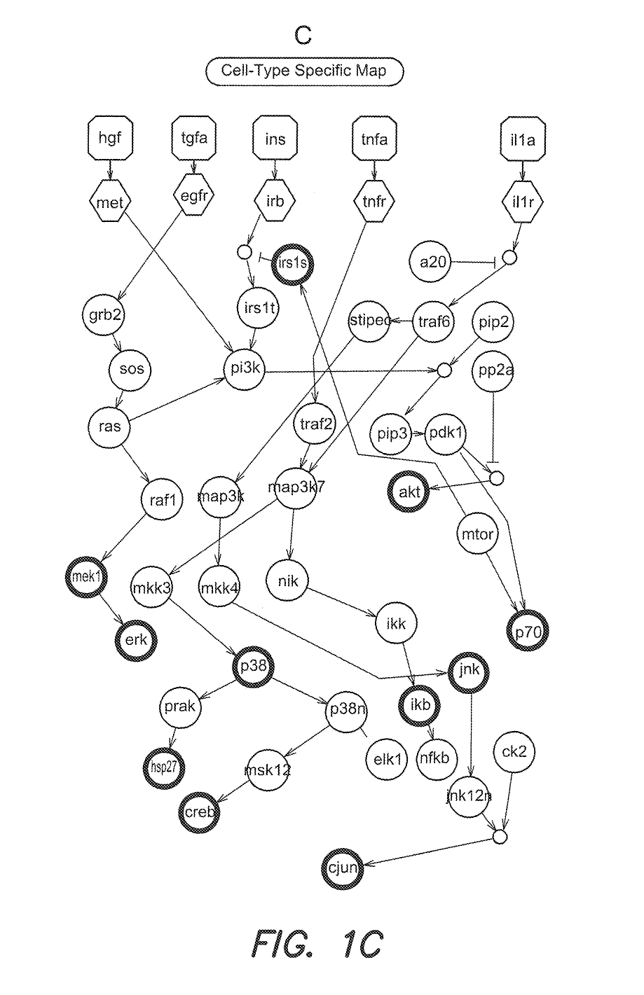

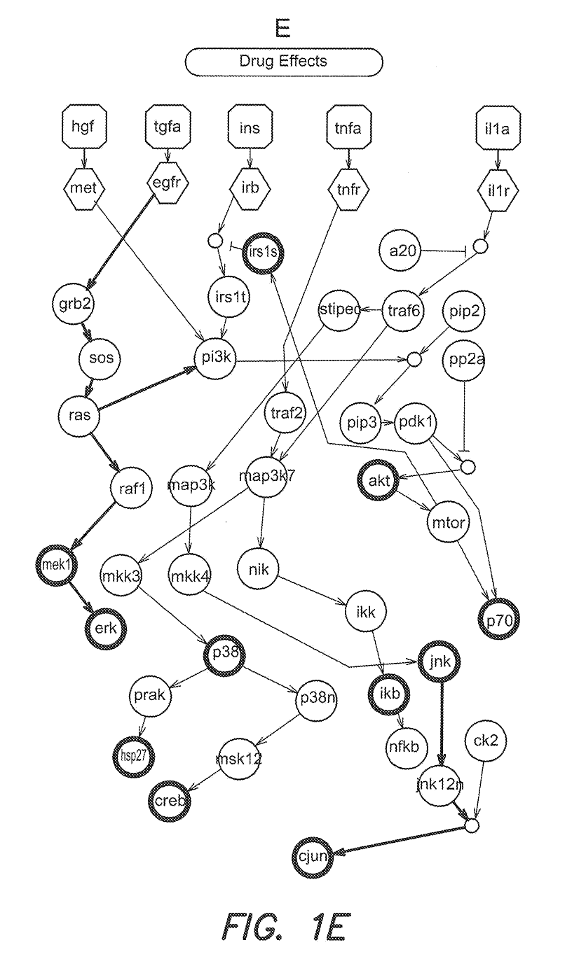

The present disclosure describes a significantly different approach to identify drug effects, in which drugs are evaluated by the alterations they cause on signaling pathways. Instead of identifying binding partners, the methods and models provided herein monitor signaling pathway alterations by assessing key signaling events, such as protein phosphorylation events, under several treatments with signaling pathway activators, for example, cytokines. An exemplary workflow is presented in FIG. 1.

### Modeling Biological Interaction Networks

Some aspects of this invention relate to the generation of biological interaction networks, for example, of signaling pathway networks. Such networks typically comprise a number of nodes, for example, molecules (e.g. proteins, peptides, nucleic acids, etc.) that are members or targets of the interaction pathway. Nodes of interaction networks are typically connected by edges representing possible interactions. In some embodiments, some or all edges within a model network are signed, which means that a specific functional attribute is assigned to a given edge. Examples of functional attributes that can be assigned to an edge include, but are not limited to binding, activation, inhibition, phosphorylation, dephosphorylation, methylation, acetylation, sumoylation, ubiquitination, and hydrolysation. In some embodiments, an interaction network is constrained to edges that are assigned a single function, for example, a single function out of a limited number of functions. In some embodiments, a network map only comprises two types of edges, for example, edges that indicate activation or inhibition, edges that indicate phosphorylation or dephosphorylation, or edges that indicate repression or activation of transcription, of a target molecule, for example, a target protein or gene. In other embodiments, a network map comprises more than two, for example, 3, 4, 5, 6, 7, 8, 9, 10, or more than 10 types of edges.

In some embodiments, a network map as provided herein comprises a plurality of nodes. In some embodiments, the network comprises a plurality of designated and/or undesignated nodes. A designated node is a node representing a molecule within an interaction network map, for example, a protein that is a member of a cellular signaling pathway, that can be or is subjected to experimental manipulation or measurement. An undesignated node is a node representing a molecule within an interaction network that can not be or is not subjected to experimental manipulation or measurement. For example, in some embodiments provided herein, a cell-specific interaction network is generated by optimizing a generic interaction network to fit experimental data obtained from cells exposed to or contacted with a stimulus or a combination of stimuli perturbing the interaction network by measuring the state or activity of one or more key members of the interaction network. A node representing a member of the interaction network that is a known target of a stimulus, for example, a receptor protein that is a target of an activating ligand used as a stimulus in this scenario, would be a designated node, because it is subject to experimental stimulation. For example, a node representing EGFR in a cellular signaling pathway network comprising an EGFR signaling pathway, in which the signaling network topology is optimized to fit data obtained from a stimulation of cells with EGF, a ligand binding EGFR and modulating its activity, is a designated node. A node representing a member of a network targeted by a small molecule agent used to obtain data for the optimization of the network, for example, a small molecule inhibitor (e.g., a kinase inhibitor), is also a designated node. Further, a node representing a molecule, for example, a protein or gene, of which data is obtained for optimizing the signaling pathway network, for example, expression level or phosphorylation level data, is also a designated node. All other nodes in a network are non-designated nodes.

Some aspects of this invention relate to an approach to biological interaction modeling, in which a generic interaction topology is provided which is then optimized to fit experimental data obtained from cells of interest. In some embodiments, the experimental data comprises data measured for a plurality of nodes within the interaction network, for example, in the case of a signaling pathway network, data measured for a plurality of proteins within the signaling pathway network. In some embodiments, the experimental data comprises multiple datasets that are obtained from cells contacted with different stimuli and/or different combinations of stimuli, that are known or suspected to perturb an interaction or a reaction within the interaction network. In some embodiments, stimuli useful for generating experimental data for network model optimization include, but are not limited to, activators and inhibitors of members of a given network model. For example, in the case of an interaction network model comprising an EGFR signaling pathway, EGF, an activating ligand of EGFR, would be a useful stimulus, as would be an inhibitor that blocks the activity of another member of the EGFR signaling pathway, for example, a MEK1 inhibitor. In this very simple example, which is for illustration purposes only and should not be construed to be limiting the invention, four sets of experimental data could be obtained: 1) from cells that are not contacted with either stimulus; 2) from cells that are contacted with EGF; 3) from cells that are contacted with MEK1 inhibitor; and 4) from cells contacted with both EGF and MEK1 inhibitor. In some embodiments, it would be desirable in this exemplary scenario to obtain measurements from a presumed or known EGFR target downstream of MEK1 activity (e.g., Erk or Creb) and, preferably, also from a presumed or known EGFR target upstream of Mek1 activity (e.g., Ras, or Mek1 itself). These datasets could then be used to optimize a generic model of the EGFR signaling pathway to represent the interactions actually present in the cells from which the data was obtained.

In some embodiments, a cell-specific biological interaction network topology, for example, a cell-specific signaling pathway topology, is generated by optimization of a generic model. In some embodiments, the generic interaction topology comprises a plurality of nodes and/or interactions that are not present in the cells of interest. This can occur, for example, if a network member represented by a node is not expressed in a cell of interest, if a required agent, substrate, or reactant is lacking in the cell, or if an agent or perturbator is present in the cell that inhibits or abolishes the respective interaction. In some embodiments, the generic network may be lacking a plurality of nodes and/or interactions that are present in the cells of interest. This can occur, for example, if an interaction between two network members has not been described in the literature or database that served as the source for the generation of the generic interaction network topology. In either case, the generic network does not accurately reflect the conditions prevailing in the cells of interest and, thus, will not fit experimental data obtained from these cells with the same goodness of fit that would be achieved with a more accurate model.

The basic concept for optimizing a generic interaction network topology to create a cell-specific interaction network topology based on experimental data, is to modify the generic topology, for example, by deleting a reaction and/or a node, or a plurality of interactions and/or nodes, to calculate the fit of the modified topology to the experimental data, and to identify the modified topology, or group of modified topologies, that exhibit the best fit to the experimental data as the optimized, cell-specific interaction network topology. One problem with this concept is, that a linear increase in the number of nodes in a given topology is related to an exponential increase in possible modified network topologies for which a fit to experimental data has to be computed. Accordingly, the use of a computationally inefficient formulation for the translation of an interaction network into a computer executable form will quickly result in time requirements for such calculations which are beyond a user-tolerable scope, or in quantities of calculations beyond today's computational capabilities. This problem is addressed by the methods and algorithms provided herein, which use highly efficient ILP formulations for interaction network modeling.

### Using ILP to Model Biological Interaction Networks

Some aspects of this invention relate to the use of integer linear programming (ILP) to model and optimize biological interaction network topologies. Linear programming (LP) algorithms typically comprise two elements: (I) an objective function and (II) a set of constraints restricting the possible solutions to the objective function. Both the objective function and the constraints are linear. Typically, an LP formulation is generated by defining the decision variables involved in a specific problem and then formulating the objective function and constraints in terms of these decision variables. ILP is a specific type of linear programming, which restricts the possible values of the decision variables to integers. ILP relies on the fundamental assumption that the problem to be solved is linear in nature and that it can either be solved or a close-to-optimal solution can be identified, for example, via heuristic methods, by using only integers as values for the decision variables. If the decision variables are further constrained to binary values, this special type of ILP is sometimes referred to as binary linear programming (BLP), which, for the purpose of this application, is included in the term ILP. A decision variable is a variable in an optimization problem that can assume a range of values which affect the quality of the solution reached. For example, in some ILP algorithms provided herein, a binary decision variable indicates whether a species is present (1) or not (0) in an experiment.

The use of ILP-based algorithms to optimize a generic interaction network topology, as provided in some embodiments of this invention, has several advantages over conventional approaches: highly sophisticated, fast, and reliable deterministic ILP solvers can be employed; the inherent linearity of the problem can be exploited; powerful heuristics are available to identify close-to-optimal solutions of ILP optimization problems in a time-efficient manner; and ILP formulations are highly scalable, which is essential for modeling more complex networks. A number of commercially available, deterministic ILP solvers suitable for use with the methods and algorithms disclosed herein are known to those of skill in the art. However, it will be appreciated that the invention is not limited to commercially available solvers, but that any suitable ILP solver can be employed.

In some embodiments, an ILP formulation for optimizing a generic biological interaction network to fit experimental data comprising actual measurements, as described in more detail elsewhere herein, is provided that comprises a primary objective function aiming to minimize the error between model predictions and actual measurements. In some embodiments, the ILP formulation further comprises a second objective function aimed at minimizing the number of possible reactions. An example of an ILP formulation comprising two exemplary objective functions as described above is provided herein as formulation (1).

Some embodiments of this invention provide methods and algorithms for the generation of cell-specific interaction network topologies by optimizing a generic network topology using ILP-based formulations to fit experimental data obtained from cells of interest. In some embodiments, as described in more detail elsewhere herein, an interaction pathway, for example, a signaling pathway, is described as a set of reactions, wherein each reaction is comprised of three corresponding index sets, namely the index set of signaling molecules, inhibitors, and “products”. In some embodiments, a signaling molecule is a molecule represented by a node in the interaction network or pathway, a reaction is represented by an edge, and a reaction product is represented by a second molecule represented by a node, or a state of such a molecule, for example, an active or inactive state, or a phosphorylation state, of a signaling protein that is a member of a signaling pathway, wherein an activity of the protein depends on its phosphorylation state. In some embodiments, the index sets relating to a reaction are all subsets of a species index set. In some embodiments, a reaction is defined to take place if and only if all reagents and no inhibitors are present. In some embodiments, if a reaction takes place, all products of the reaction are formed. In some embodiments, reactions without products and/or reactions with neither reagents nor inhibitors are excluded during the optimization step.

In some embodiments, the set of species is known. For example, in some embodiments, the interaction network topology is a signaling pathway topology that comprises a number of signaling molecules, for example, signaling molecules the activity of which depends on their phosphorylation status and that can be activated (e.g. phosphorylated) or inactivated (e.g., dephosphorylated or otherwise inhibited) by other signaling molecules of the signaling pathway topology or by extrinsic stimuli, (e.g. receptor ligands, small molecule inhibitors, etc.). However, in some such embodiments, the set of reactions that take place in a cell of interest is not known. Rather, only a superset of potential reactions is postulated in the generic interaction network topology. In some embodiments, the goal of an ILP formulation as provided herein is to find an optimal set of reactions out of such a superset of potential reactions. In other words, the goal of an ILP formulation as provided herein is to identify a cell-specific interaction network that represents a modified version of the generic interaction network, by generating a plurality of modified interaction networks and determining which of these most accurately reflects experimental data obtained from the cells of interest. In some embodiments, the ILP formulation employs binary decision variables indicating if a reaction is possible (1) or not (0), wherein 1 indicates that a connection between two nodes is present, whereas 0 indicates that such a connection is not present.

In some embodiments, a set of constraints is included in an ILP formulation for the optimization of the generic model to fit the experimental data. Such constraints depend on the nature of the network to be modeled as well as on the type of interactions present in the modeled network and the type of cell of interest. Exemplary constraints and their corresponding ILP formulations are described in detail in the Example section. Exemplary, non-limiting constraints include constraints limiting the number of combinations of connectivities; constraints defining that a reaction only takes place, if it is possible; constraints defining that a reaction can only take place, if all reagents are present; constraints defining that a reaction can only take place if no inhibitor is present; constraints that define that if a reaction takes place, all required reagents are present; constraints that define that if a reaction takes place, all inhibitors of the reaction are absent; constraints that define that, if a reaction occurs in which a species is a product, the species is formed; constraints allowing for the generation of the same product from a plurality of reactions; constraints that define that, if none of a plurality of reactions occurs, of which a species is a product, then the species is not formed. Other constraints useful for modeling specific biological interaction networks as well as their ILP-based formulations will be apparent to those of skill in the art and the invention is not limited in this respect. In some embodiments, an ILP formulation is provided for modeling biological interaction networks that comprises binary decision variables for the manipulated species. In some embodiments, decision variables for manipulated species are replaced by constants or removed.

In some embodiments, a generic interaction network is modified, for example, by deleting species and/or reactions from the generic network, and a plurality of modified interaction networks produced by such modifications is then evaluated for their goodness of fit in regard to experimental data. One possibility for identifying the interaction network that exhibits the best fit to the experimental data would be to enumerate all possible solutions, i.e. all possible modified interaction networks, calculate the fit of each of them, and pick the one, or the ones that show the best fit. However, this strategy is feasible only for simple generic interaction networks that can give rise to only a limited number of modified interaction networks. In some embodiments, for example, in embodiments where the interaction network comprises more than 10, more than 20, more than 50, more than 100, more then 200, more than 500, or more than 1000 nodes, an optimal or close-to-optimal modified interaction network is identified by using heuristics, branch-and-bound and/or branch-and-cut strategies. In some embodiments, the ILP optimization problem is solved by employing heuristics to identify a close-to-optimal solution in a reduced amount of time as compared to the identification of the optimal solution to the optimization problem. Branch-and-bound and branch-and-cut are art-recognized terms referring to implicit enumeration techniques, in which many possible solutions will be skipped during enumeration as they are known to be non-optimal. In some embodiments, the optimization problem is solved by using a commercially available ILP solver that employs heuristics, branch-and-bound, and/or branch-and-cut strategies.

In a set of proof-of-principle experiments, 13 key phosphorylation events were measured under more than 50 different conditions generated by the combinatorial treatment of stimuli and selective inhibitors of signaling pathway interactions using bead-based multiplexed assays [17]. Based on the signaling response and an a-priori set of possible reactions (herein referred to as a generic signaling pathway topology), a cell-type specific pathway topology was generated using an efficient Integer Linear Programming (ILP) optimization formulation. This approach builds upon the Boolean optimization approach proposed in [18]. The ILP is solved using standard commercial software packages to guaranteed global optimality (within a user-defined, numerically small tolerance). To evaluate drug effects, cells were subjected to the same stimuli in the presence of drugs and the alterations of the same key phosphorylation events were tracked. Then, the ILP formulation was re-applied without a-priori assumption of the drug target, and the changes in the pathway topology with and without drug presence were assessed.

To demonstrate this approach, a generic signaling pathway map was constructed and optimized to fit the phosphoproteomic data of the transformed hepatocytic cell lines HepG2. Then, the effects of four drugs on the optimized, cell-specific signaling pathway topology were identified using an ILP strategy as provided herein. The four drugs investigated included the dual EGFR/ErbB-2 inhibitor Lapatinib [19], two potent EGFR kinase inhibitors, namely Erlotinib [20] and Gefitinib [21], and the “dirty” Raf kinase inhibitor Sorafenib [22]. The attribute “dirty” in this context refers to the non-specific character of this kinase inhibitor, which binds and inhibits multiple kinase targets. The main target effect of these 4 drugs were identified using this approach. Further, several unknown but equally active off-target effects were identified. For example, in the case of Gefitinib, a previously unknown inhibition of cJUN in the IL1α pathway was detected.

In contrast to previously developed techniques, the methods for the generation of cell-specific signaling pathway topologies and the characterization of drug effects on cellular interaction networks, as disclosed herein, are based on actual observations, for example, of the actual effect on phosphorylation events, carefully spread into a signaling pathway network. Theoretically, this approach can be applied to any type of intracellular and/or extracellular perturbations, for example, to ATP-based and allosteric kinase inhibitors, RNAi, shRNA, gene alterations et cetera. A major advantage of the methods and algorithms disclosed herein is that the ILP-based methods are computationally more efficient and faster to perform than methods based on current algorithms for pathway optimization [18]. Accordingly, the instantly disclosed ILP-based methods allow for the modeling of signaling pathway topologies having a degree of complexity that is beyond the computationally feasible scope of current modeling methods. The methods and algorithms disclosed herein allow for the identification of the main drug effects as well as unknown off-target effects in areas of pathways constrained between the activated receptors and the measured phosphorylated proteins.

The ability to model complex signaling pathway topologies in a computationally efficient is advantageous, because the more complex the modeled signaling pathway topology is, the more complete the characterization of a drug or other perturbator of such a topology, or, in other words, the higher the chances to detect an effect, for example, an unanticipated off-target effect, of a drug on a cellular interaction network. In some embodiments, the methods and algorithms described herein are used to model a cellular interaction network, for example, a signaling pathway topology, comprising at least 10, at least 20, at least 30, at least 40, at least 50, at least 60, at least 70, at least 80, at least 90, at least 100, at least 150, at least 200, at least 250, at least 300, at least 400, at least 500, at least 600, at least 700, at least 800, at least 900, at least 1000, at least 2000, at least 3000, at least 4000, at least 5000, at least 6000, at least 7000, at least 8000, at least 9000, or at least 10000 nodes and/or reactions. In some embodiments, the methods and algorithms disclosed herein are used to model an entire signaling network, for example, by integrating whole-kinome data, whole-phosphoproteome data, whole-proteome data, or whole-transcriptome data, into logic based models using methods and algorithms as described herein.

In some embodiments, a fast and unbiased characterization of drug effects with methods and algorithms provided herein is useful to elucidate the mechanism by which a particular drug effect is mediated, to analyze and/or predict the efficacy and toxicity of a drug in a given clinical scenario, and/or to select the most effective drug for the treatment of a specific disease by matching aberrant signaling topologies in diseased cells to specific drugs having the highest normalizing effect on such topologies.

Modeling complex signaling pathway topologies in a computationally efficient manner by using the methods and algorithms described herein allows for the unbiased assessment and detection of signaling pathway alterations in a population of cells of interest, for example, in a population of cells that were exposed to a drug or a population of cells in an aberrant or diseased state.

### Generic Interaction Network Topologies

Some embodiments of this invention provide methods and algorithms for the analysis and modeling of cell interaction networks, for example, of cellular signaling topologies, and the detection of drug effects on such networks. In some embodiments, a generic network map comprising a set of nodes and edges linking the nodes and representing reactions is generated or provided. In some embodiments, the edges represent Boolean gates, for example, AND, OR, or AND NOT gates. In some embodiments, the generic interaction network map is curated from literature, for example, by mining literature for descriptions of pathway interactions and assembling the generic map by compiling the information found in the literature into a pathway map. Sources of literature disclosing interactions relevant for the curation and generation of generic interaction networks are well known to those of skill in the art and include, for example, the National Center of Biotechnology Information (NCBI) database, accessible at ncbi.org.

In some embodiments, the generic interaction network map is generated from an interaction network database. Interaction network databases providing information useful for the generation of generic interaction maps, for example, databases providing signaling pathway interactions or protein-protein or protein-substrate interactions, are well known to those of skill in the art, and include, for example, the Pathway Interaction Database (PID, accessible at pid.nci.nih.govf), the KEGG pathway database (genomejp/kegg/), the Biocarta database (biocarta.com), the Reactome database (reactome.org), the BioCyc database, (biocyc.org) the PANTHER database (pantherdb.org), the GenMAPP/WikiPathways database (wikipathways.org), the Science STKE database (stke.sciencemag.org), and the Lipid MAPS database (lipidmaps.org). In some embodiments, a generic signaling pathway map comprises actual observations, for example, observations of interaction in similar or different cells or cell types as the cells or cell types of interest.

In some embodiments, a generic interaction network, for example, a generic pathway topology is generated or provided that is focused on a specific signaling pathway, for example, on interleukin signaling topologies. In other embodiments, it is preferable for the generic interaction topology to be as expansive as possible, for example, to cover as many different signaling pathways and as many members of such pathways as possible. In some embodiments, the generic interaction network topology comprises all known cellular molecules involved in cellular signaling. In some embodiments, the generic signaling pathway topology comprises all known cellular molecules of a specific type, for example, kinases, phosphatases, phosphoproteins, etc., involved in cellular signaling. In some embodiments, such global generic interaction network topologies form the basis for a global, unbiased characterization of drug effects and/or disease states on the interaction networks in specific cells of interest.

### Cell-Specific Interaction Network Topologies

In some embodiments, a cell-specific interaction topology, for example, a cell-specific signaling pathway map or model, is generated by constraining a generic topology to fit actual observations of interactions, for example, observations of signaling pathway activity, for example, measurements of key phosphoprotein signals under different experimental conditions. In some embodiments, the observations used to optimize a generic interaction topology are restricted to observations made in a cell, cell type, cell population, or tissue type of interest, for example, in kidney cells, lung cells, liver cells, brain cells, blood cells, spleen cells, or tumor cells. In some embodiments, the fitting of a generic interaction map to a set of actual observations is performed via an Integer Linear Program (ILP) formulation. In some embodiments, the optimization of the generic interaction map by fitting to actual observations is carried out by using standard ILP solvers, a procedure that drastically outperforms previous fitting schemes.

In some embodiments, a method of generating a cell-specific interaction network topology using ILP is provided. In some embodiments, the interaction network topology is a cellular signaling pathway topology. In some embodiments, the method comprises the steps of (a) providing a generic interaction network topology, for example, a generic cellular signaling pathway topology; (b) measuring a quantitative baseline value for a plurality of nodes within the generic interaction network topology; (c) measuring a quantitative value for each of the plurality of nodes measured in (b) in the presence of a stimulus known to perturb the generic interaction network topology; optionally, (d) comparing the values measured for each of the plurality of nodes in (b) and in (c); and (e) modifying the generic interaction network topology and using an ILP algorithm to identify a modified generic interaction network topology or group of such topologies that best fit the values determined in (b) and (c) as the cell-specific interaction network topology.

In some embodiments, the generic topology comprises a signaling pathway, wherein the signaling pathway comprises a plurality of nodes representing signaling molecules, for example, proteins that are members of the signaling pathway, and wherein the nodes are connected via edges representing reactions within the pathway, for example, activating or inhibiting reactions, such a phosphorylation, dephosphorylation, binding, or ubiquitination.

In some embodiments the baseline value for a node is a value determined in the cell of interest for the node in the absence of a stimulus or inhibitor affecting the pathway. The nature of the value is, of course, dependent on the measurement employed. For example, the value can be an expression level of a gene, mRNA, or protein, or the activity, for example, the kinase or phosphatase activity, of a protein, or the state, for example, the phosphorylation state, or the inclusion in or exclusion from a complex, etc. Methods for determining such values are well known to those in the art, and include, PCR-based methods (e.g., PCR, RT-PCR), hybridization-based methods (e.g., expression arrays, northern blot), sequencing-based methods (e.g. massive parallel sequencing methods), immunodetection-based methods (e.g., western blot, ELISA, protein arrays, phospho-specific antibody arrays, etc.) and other methods that will be apparent to those of skill in the art. It should be noted that the invention is not limited in this respect and that any suitable measurement of relevant parameters related to a molecule represented by a node in the specific interaction network can be employed. To give a non-limiting example, the baseline value for a direct target molecule of EGFR, e.g. Grb2, in a signaling pathway topology could be determined by measuring the phosphorylation state of Grb2 in the absence of any activating stimulus of EGFR, for example, in the absence of EGF.

In some embodiments, the step of (c) involves measuring a quantitative value for each of the plurality of designated nodes measured in (b) in a cell of the same cell type or state as employed in step (b) contacted with a stimulus known to perturb the generic signaling topology. The term stimulus refers to any extrinsic molecule or condition that is known to perturb the generic topology, for example, by activating or inhibiting an interaction comprised within the generic topology. In some embodiments, the stimulus is a molecule, for example, a ligand of a receptor that is represented by a node in the generic topology. For example, in a signaling pathway topology comprising an EGFR signaling pathway, where EGFR is represented as a node within that pathway, EGF, which activated EGFR signaling, is a stimulus, because it perturbs the basic state of EGFR, as measured in cells not contacted with EGF. This can easily be confirmed by contacting the cells with EGF, measuring the activity of a member of the EGFR pathway, for example, the phosphorylation level of Grb2 or Ras, and comparing the value measured in the presence of EGF to that in the absence of EGF, (e.g., as determined in step (b)). If EGF perturbs the EGFR pathway, then the activity of the EGFR pathway member in the presence of EGF should be significantly different from that measured in the absence of EGF.

In some embodiments, step (d) can be employed to formulate the observed differences in a way that can be executed in order to facilitate model optimization in step (e). For example, this step can comprise, in some embodiments, expressing the values obtained in (b) and (c) as a ratio, or other function representing the difference, if any, of the values of (b) and (c).

In some embodiments, the optimization step (e) comprises a step of modifying the generic interaction topology. In some embodiments, the step of modifying comprises generating a topology by deleting a species and/or a reaction comprised in the generic topology. In some embodiments, step (e) comprises generating a plurality of modified interaction topologies and using an ILP algorithm to identify the modified topology, or group of such topologies, that best fit the experimental data obtained in (b) and (c) as the cell-specific interaction topology. In some embodiments, the ILP optimization problem of identifying the modified interaction topology exhibiting the best fit to the experimental data is solved using a commercially available ILP solver. In some embodiments, the ILP optimization problem is solved by determining a close-to-optimal solution, for example, by employing a heuristic strategy. In some embodiments, the ILP optimization problem is solved by skipping solutions (modified interaction networks) that are known to be suboptimal. In some embodiments, the ILP optimization problem is solves by employing a branch-and-cut and/or a branch-and-bound strategy.

In some embodiments, the ILP algorithm defines a pathway within the topology, for example a signaling pathway within a signaling pathway topology, as a set of reactions and species, wherein each species is represented by a node in the pathway topology and each reaction is represented by an edge in the pathway topology. In some embodiments, a reaction is defined by three index sets: (1) a set of signaling molecules, (2) a set of perturbations, and (3) a set of reaction products. In some embodiments, the perturbations include inhibitors and activators, for example, inhibitors or activators of a reaction, for example, a phosphorylation reaction, a dephosphorylation reaction, a binding interaction, transcription, translation, expression, or degradation. In some embodiments, the inhibitors include small-molecule inhibitors, for example, small molecule kinase inhibitors, phosphatase inhibitors, inhibitors of other enzymes, for example, other enzymes involved in signaling or metabolic pathways. Small molecule inhibitors of numerous enzymes and signaling molecules are well known in the art and the invention is not limited in this respect. Any small molecule inhibitor known to act upon a molecule, for example, a protein, represented in a generic biological interaction network as provided herein is useful as a perturbator of such a network. In some embodiments, ligand inhibitors, inhibitory drugs, repressors of gene expression, and/or RNAi agents are used as perturbations. Such agents are well known to those of skill in the art and the invention is not limited in this respect. RNAi agents include, but are not limited to siRNAs, shRNAs, antisense RNAs, and miRNAs, both naturally occurring and synthetic. In some embodiments, the index sets provided under (1), (2), and (3) above are subsets of the species index set.

In some embodiments, at least one value in the set of reaction products represents a phosphorylation level of a protein comprised in the set of signaling molecules. In some embodiments, measured values comprised in the experimental data comprise phosphorylation levels of signaling molecules. Methods for measuring phosphorylation levels of biological molecules are well known to those of skill in the art and include, for example, immunoassays employing phospho-specific antibodies, which only recognize either the phosphorylated or the unphosphorylated form of a given protein, or using Mass-Spectrometry-based methods. Some methods useful for the determination of phosphorylation levels are described herein and others will be apparent to the skilled artisan, as will be the fact that this invention is not limited in this respect.

In some embodiments, the ILP algorithm used to model a biological interaction network topology and/or to identify the modified topology that best fits the experimental data comprises a constraint defining that a reaction takes place only if all reagents and no inhibitors are present. For example, if a reaction in a signaling pathway topology requires EGFR and EGF, and can be inhibited by an EGFR inhibitor (for example, Lapatinib), the reaction will only take place if EGFR and EGF are present and Lapatinib is absent, but not if only EGF is present (but EGFR is absent), or if both EGFR and EGF are present but Lapatinib is present as well. In some embodiments, the ILP algorithm comprises a constraint defining that if a reaction takes place, all products are formed. In some embodiments, the ILP algorithm comprises a constraint excluding reactions without products and/or reactions with neither reagents nor inhibitors. In some embodiments, this constraint boosts computational efficiency of network modeling by eliminating reactions that cannot be manipulated, or that do not result in the formation of a product, and are thus inconsequential for the modeled network.

In some embodiments, the ILP algorithm comprises binary decision variables indicating whether a reaction is possible or not. In some embodiments, using binary decision variables further improves computational efficiency. In some embodiments, experimental data values are translated into binary values as well. For example, an experimental data point is assigned a value of 1, representing an “on” or “present” state, if the experimental value is above a certain threshold and a value of 0, representing an “off” or “absent” state, if the experimental value is below that threshold. In embodiments, where the experimental data comprises protein phosphorylation data, a protein may be assigned a 1, representing “phosphorylated”, if more than half of the amount of total protein is determined to be phosphorylated and a value of 0, or “unphosphorylated”, if less than half of the total protein is phosphorylated. In some embodiments, the ILP algorithm comprises integer decision variables. In some embodiments, the ILP algorithm comprises only integer decision variables. In some embodiments, the ILP algorithm comprises binary decision variables. In some embodiments, the ILP algorithm that comprises only binary decision variables. In some embodiments, at least one of the ILP decision variables is relaxed to continuous. In some embodiments, the relaxation of the at least one of the ILP decision variables does not alter the feasible set. In some embodiments, at least one of the ILP decision variables is a real decision variable. In some embodiments, a reaction that is not possible is represented as an edge not being present, and a reaction that is possible is represented as an edge being present.

In some embodiments, step (e) comprises determining the fit of the generic topology to the observed data; modifying the generic topology by adding and/or removing reactions to generate a plurality of modified topologies; determining the fit of each modified topology so created or of a subset of the plurality of modified topologies so created to the observed data; and selecting the modified topology exhibiting the best fit to the experimental data as the cell-specific topology. In some embodiments, determining the fit of the topology to the experimental data comprises determining the goodness of fit of the topology. In some embodiments, the topology exhibiting the best goodness of fit is selected as the cell-specific topology. In some embodiments, determining the goodness of fit comprises calculating the percentage error. In some embodiments, the modified topology or group of modified topologies with the best fit or a close-to-optimal fit is/are selected as the cell-specific topology or group of topologies.

In some embodiments, the method for generating a cell-specific interaction network topology further comprises (f) optimizing the cell-specific topology to minimize the number of nodes and/or possible reactions without worsening the fit of the topology to the experimental data. In some embodiments, this step eliminates reactions and molecules within the generic topology that cannot be measured and/or manipulated and reactions or species that have no predictive value. The status of such molecules or reactions cannot be validated or used to predict or analyze topology alterations and is, therefore, of little or no value for the purposes of some embodiments of this invention.

In some embodiments, the stimulus known to perturb the generic signaling topology comprises an agent activating or inhibiting the activity of a signaling molecule within the signaling topology, or a combination of such agents. In some embodiments, a number of stimuli, e.g., activators and inhibitors of pathways comprised in a generic topology, are identified and experimental data for topology optimization is generated by contacting a cell population of interest with the different stimuli and/or combinations of the different stimuli, as described in more detail elsewhere herein. In some embodiments, the stimulus comprises a plurality of agents, each agent individually activating or inhibiting the activity of a signaling molecule within the signaling topology. For example, in some embodiments, the stimuli comprise a number of activators and a number of inhibitors of molecules comprised in the generic pathway. In some embodiments, experimental data is obtained from a population of cells of interest contacted with each of the activators and inhibitors alone, and with combinations of activators, combinations of inhibitors, and/or combinations of activator(s) and inhibitor(s). If a plurality of activators and inhibitors is used to generate the experimental data, stimuli may include a single activator, a single inhibitor, a combination of a single activator with a single inhibitor, a combination of activators, a combination of inhibitors, a combination of a plurality of activators and a single inhibitor, a combination of a plurality of inhibitors and a single activator, and a combination of a plurality of activators with a plurality of inhibitors.

In some embodiments, the step of (c) comprises a plurality of separate measurements of cells contacted with different stimuli known to perturb the generic signaling topology, for example, as provided immediately above. In some embodiments, the different stimuli comprise a ligand and/or a small molecule inhibitor of a signaling molecule, or different signaling molecules, comprised in the generic signaling topology, or different combinations of such ligands and/or inhibitors. As will be apparent to the skilled artisan, any agent able to perturb a given interaction topology is suitable to be used as a stimulus. Agents that directly interfere with the readout used to obtain the experimental data are preferred in some embodiments. For example, in some embodiments, in which the experimental data comprises protein phosphorylation levels, preferred stimuli may be those that interfere with protein phosphorylation, for example, kinase and/or phosphatase inducers and/or inhibitors. In some embodiments, the ligands are selected from the group of growth factors, cytokines, intercellular signaling molecules, and intracellular signaling molecules. In some embodiments, the ligands are selected from the group comprising Tumor necrosis factor alpha (TNF-alpha), Interleukin 1-alpha (IL1-alpha), hepatocyte growth factor (HGF), insuling (INS), and Transforming growth factor-alpha (TGF-alpha). In some embodiments, the small molecule inhibitors are selected from the group comprising PI3K, MEK, p38, cMET, and EGFR inhibitors. Other suitable substances that can be used as a stimulus according to aspects of this invention, either alone or in combination with other substances, will be apparent to those of skill in the art. It should be appreciated that the invention is not limited in this respect.

In some embodiments, the generic interaction topology comprises at least 10, at least 20, at least 30, at least 40, at least 50, at least 60, at least 70, at least 80, at least 90, at least 100, at least 150, at least 200, at least 250, at least 300, at least 400, at least 500, at least 600, at least 700, at least 800, at least 900, at least 1000, at least 2000, at least 3000, at least 4000, at least 5000, at least 6000, at least 7000, at least 8000, at least 9000, or at least 10000 nodes. In some embodiments, the generic signaling pathway topology represents part of or all of the kinome and/or phosphoproteome of the cell.

### Characterization of Drug Effects on Cell-Specific Interaction Network Topologies

In some embodiments, once a cell-specific topology is known, for example, based on optimization of a generic interaction network to fit cell-specific interaction data, the same interaction data is analyzed in the presence of a drug to generate a cell-specific, drug-induce topology. For example, in some embodiments, once a cell-specific signaling pathway topology of a specific cell type is known based on the analysis of key phosphoprotein signals within a generic signaling pathway topology in the absence of a candidate drug, the same key phosphoprotein signals are analyzed in the presence of the drug. The cell specific topology is then re-optimize to fit the data obtained in the presence of the drug to reveal drug-induced topology alterations.

For example, as described in more detail in the Example section, a cell-specific topology for the hepatocytic cell-line HepG2 was generated from a generic topology based on fitting the generic topology to measurements of 13 key phosphoproteins within the generic topology under 10 different experimental conditions including combinatorial treatments with pathway activators and inhibitors. Once the HepG2 cell-specific signaling pathway map was generated, the effects of 4 drugs: 3 selective inhibitors for the Epidermal Growth Factor Receptor (EGFR) and a non-selective drug, was analyzed by fitting the HepG2 cell-specific signaling network topology to data of the same 13 key phosphoproteins obtained from HepG2 cells under the same experimental conditions and contacted with the respective drug. A comparison of the cell-specific signaling topologies to the drug-induced signaling topologies revealed drug-induced topology alterations. Effects easily predictable from the drugs' main target (EGFR inhibitors block the EGFR pathway) were detected this way, illustrating the validity and accuracy of this approach. However, unanticipated effects of the drugs were also uncovered, attributable to either drug promiscuity or the cell's specific topology. An interesting finding was that the selective EGFR inhibitor Gefitinib inhibits signaling downstream the Interleukin-1alpha (IL1α) pathway; an effect that cannot be extracted from binding affinity-based approaches. Accordingly, the methods and algorithms provided herein represent an unbiased approach to identify drug effects. This methodology is applicable to small and medium sized pathways, but is also scalable to larger topologies, including global topologies, with any type of signaling interventions (small molecules, RNAi, etc). The methods and algorithms provided herein can reveal drug effects on cellular interaction pathways, the cornerstone for identifying mechanisms of a drug's efficacy.

In some embodiments, an unbiased, phosphoproteomic-based approach to generate a cell-specific interaction network topology and/or to identify a drug's effect(s) on such a topology by monitoring drug-induced topology alterations is provided. In some embodiments, the interaction network topology is a signaling pathway topology. In some embodiments, a method for identifying an effect of a drug on a molecular signaling pathway in a cell using integer linear programming is provided. In some embodiments, the method comprises the steps of (a) providing a cell-specific topology (b) determining drug-induced signaling topology alterations of the cell-specific topology, thus creating a cell-specific, drug-induced topology, and (c) comparing the cell-specific topology to the cell specific, drug-induced topology to determine which interactions in the cell-specific topology are affected by the drug.

In some embodiments, the step of (b) comprises a step of (i) contacting a cell of the same cell type as the cells to which the cell-specific topology provided in (a) applies with a drug known or suspected to perturb at least one reaction in the topology provided in (a). In some embodiments the drug is a known drug. In some embodiments, the drug is approved for the treatment of a human disease. In some embodiments, the drug is a candidate drug, for example, a candidate drug from a library of drugs. In some embodiments, the drug is a small compound. In some embodiments, the drug is a peptide or protein. In some embodiments, the drug is a ligand of a cellular receptor. In some embodiments, the drug is a binding agent, for example, an antibody or an antigen-binding fragment of an antibody, an aptamer, or an adnectin In some embodiments, the drug is a ribozyme or DNAzyme. In some embodiments, the drug is an RNAi agent, for example, an siRNA, an shRNA, an antisense RNA, or a microRNA. In some embodiments, the cell is contacted with the drug in the absence and/or in the presence of the stimulus known to perturb the signaling pathway. In some embodiments, the drug is a kinase inhibitor. Kinase inhibitors are well known to those of skill in the art and numerous kinase inhibitors have been and are being developed for clinical use. Kinase inhibitors are able to disrupt interactions that depend on kinase activity and there is a large body of evidence that aberrant kinase activity contributes to or causes human disease. Exemplary kinase inhibitor drugs include, but are not limited to VEGF kinase inhibitors (e.g. Bevacizumab, Ranibizumab, Pegaptanib), VEGFR2 kinase inhibitors (e.g., E7080, Pazopanib), EGFR/Erb2 kinase inhibitors (e.g., BIBW 2992), Erb1 kinase inhibitors (e.g., Cetuximab), Bcr-Abl kinase inhibitors (e.g., Imatinib, Nilotinib), Erb2 kinase inhibitors (e.g., Trastuzumab), EGFR kinase inhibitors (e.g., Gefitinib, Erlotinib, Lapatinib, Panitumumab), and multi-target kinase inhibitors (e.g., Sorafenib, Dasatinib, Sunitinib). It will be appreciated that the invention is not limited in this respect and that the effect of any drug, for example, any kinase inhibitor, on a biological interaction network within a cell can be analyzed using the methods and algorithms provided herein.

In some embodiments, the step of (b) comprises a step of (ii) measuring a quantitative value for a plurality of designated nodes in the cell-specific topology in a cell contacted with the drug in the absence and/or the presence of the stimulus known to perturb the cell-specific signaling topology. In some embodiments, the quantitative value is a value representing an expression level, a concentration, an activity, for example, an enzymatic activity (e.g., a kinase or phosphatase activity) or the state of a molecule, for example, a phosphorylation state of a protein.

In some embodiments, the step of (b) comprises a step of (iii) generating a cell-specific, drug-induced topology by modifying the cell-specific topology and using an ILP algorithm to identify a modified cell-specific topology or group of topologies that best fit(s) the experimental values measured in (b)(ii). In some embodiments, a modified cell-specific topology that best fits is a topology exhibiting the best fit to the experimental data. In some embodiments, a modified topology that best fits is a topology that exhibits a close-to-optimal fit to the experimental values.

In some embodiments, the method further comprises a step of (c) comparing the cell-specific topology provided in (a) to the cell-specific, drug-induced topology generated in (b)(iii). In some embodiments, if a reaction is present in the cell-specific topology but absent in the cell-specific, drug-induced topology, the reaction is indicated to be inhibited by the drug; (ii) if a reaction is absent in the cell-specific topology but present in the cell-specific, drug-induced topology, the reaction is indicated to be activated by the drug; and/or (iii) if a reaction is either absent or present in both the cell-specific and the cell-specific, drug-induced topology, the reaction is indicated to not be affected by the drug.

In some embodiments, the ILP algorithm is the ILP algorithm as described elsewhere herein, for example, the ILP algorithm as described in the Example section or the section relating to the generation of the cell-specific interaction network topology above. In general, the ILP algorithm used for determining drug effects on interaction networks are similar to those used for determining cell-specific topologies from generic topologies. This is true, at least in part, because the concept of the problem is similar: modified versions of a provided interaction network topology are generated and evaluated regarding their fit to experimental data.

The modified topology with the best fit is then identified as the topology most accurately reflecting the actual state of the interaction network topology in the given cell under the given circumstances (e.g., in the presence or the absence of a drug). However, it will be appreciated by those in the art that there will be some differences in ILP algorithms based on their specific purposes within the scope of this invention. For example, some ILP algorithms as provided herein for the generation of cell-specific topologies comprise constraints defining that a given inhibitor inhibits interactions downstream of its primary target (e.g. a PI3K inhibitor inhibits interactions downstream of PI3K). In some embodiments, such a priori inhibitor constraints are removed from the ILP algorithm used for determining the effects of a candidate drug on a cell-specific signaling topology, even, in some cases, where the primary target of a drug is known. This strategy allows for truly unbiased assessment of the effects a drug exerts on a biological interaction topology, which is a key to understanding drug function and efficacy as well as undesired drug side effects.

### ILP-Based Interaction Network Models in Personalized Medicine

Some aspects of this invention relate to the application of ILP based biological interaction network modeling approaches to the selection and application of clinical interventions. In some embodiments, a method for selecting and/or administering a drug targeting a molecule or reaction within a cellular interaction network is provided. In some embodiments, the method is for the treatment of a subject diagnosed with a disease based on a cellular interaction topology assessment. In some embodiments, the cellular topology assessment is a signaling pathway topology assessment. In some embodiments, the method comprises a step of (a) generating or obtaining a cell-specific signaling topology from a cell population obtained from a subject. In some embodiments, the subject is a human subject. In some embodiments, the subject is a non-human mammal, a non-human primate, a mouse, a rat, a rabbit, a hamster, a cow, a sheep, a goat, a pig, a horse, a dog, or a cat. In some embodiments, the subject has been diagnosed with a disease. In some embodiments, the subject has been diagnosed with a disease for the treatment of which a plurality of different drugs is available. In some embodiments, the plurality of drugs comprises drugs that are known or suspected to have different effects on cellular interaction networks, for example, on cellular signaling topologies typically found in subjects having a disease treatable with the drug in question. In some embodiments, the cell population obtained from the subject comprises cells in a diseased state. For example, in some embodiments, the cell population comprises malignant cells. In some embodiments, a cell-specific topology is generated for the population of cells obtained from the subject by using an integer linear programming optimization scheme, for example, an ILP scheme as described in more detail elsewhere herein or otherwise provided by aspects of this invention.

In some embodiments, the method further comprises a step of (b) comparing the cell-specific signaling topology from the subject to a reference signaling topology. In some embodiments, (i) if a signaling molecule and/or reaction is absent in the reference topology and present in the topology of the subject, then treatment of the subject with a drug that inhibits the signaling molecule and/or reaction is indicated. In some embodiments, (ii) if a signaling molecule and/or reaction is present in the reference topology and absent in the topology of the subject, then treatment of the subject with a drug that activates the signaling molecule and/or reaction is indicated. For example, if a subject is diagnosed with a breast cancer, for which the administration of an EGFR kinase inhibitor is indicated, a population of cells comprising breast cancer cells is obtained from the subject, according to methods provided herein, and a cell-specific signaling pathway topology is generated from the population of cells, using methods and algorithms as described in more detail elsewhere herein. If the cell-specific topology indicates that EGFR signaling is abnormally increased, then administration of an EGFR inhibitor, for example, of Lapatinib, Gefitinib, or Erlotinib, to the subject is indicated. If the cell-specific topology does not show an aberrant increase of EGFR signaling, then administration of an EGFR inhibitor is not indicated, or may even be contraindicated. If the cell-specific topology shows that not only EGFR signaling, but also c-Jun signaling is abnormally increased in the diseased cells obtained from the subject, then administration of Gefitinib, which affects c-Jun signaling as well as EGFR signaling, as described elsewhere herein, is preferable over the administration of Lapatinib and Erlotinib, which do not affect c-Jun signaling.

Accordingly, some aspects of this invention provide methods to characterize drug effects on biological interaction networks and methods of using this information to match available drugs to aberrant interaction topologies detected in diseased subjects. This allows for the selection and/or administration of the most effective treatment available, thus increasing the chance of a desirable clinical outcome and avoiding incurring unnecessary side effects of drugs tat are administered even though they do not affect an aberrant pathway in a cell or drugs that are administered to subjects the diseased cells of which do not show an alteration in the interaction pathway targeted by the drug.

In some embodiments, a method is provided that comprises selecting a drug to be administered based on matching an interaction topology of a diseased cell population to the effect a drug has on biological interactions in the specific cell type. In some embodiments, the interaction topology of the cells derived from the subject and the drug effects on such an interaction topology are determined with methods and algorithms provided herein. In some embodiments, the method provided further comprises a step of (c) administering the selected drug to the subject. In such embodiments, the drug is administered to the subject in an amount sufficient to effect a drug-induced alteration in a drug-targeted interaction, for example, an inhibition of a kinase-mediated phosphorylation reaction, In some embodiments, the drug is administered in an amount sufficient to inhibit or activate the drug's target molecule and/or reaction.

In some embodiments, the cell population is derived from a diseased tissue of the subject. In some embodiments, the cell population is further processed to enrich for cells in a diseased state, for example, in some embodiments, a tumor biopsy may be processed to generate a population of cells enriched for cells derived from the tumor, and not from surrounding healthy tissue. Methods for enriching for cells in a diseased state are well known to those of skill in the art and include, for example, mechanical separation methods, cell sorting methods based on surface marker expression, and culturing methods favoring diseased over healthy tissue derived cells. In some embodiments, the diseased tissue is malignant tissue. In some embodiments, the diseased tissue is breast cancer, lung cancer, or colorectal cancer tissue.

In some embodiments, the drug is chosen from a group of drugs. In some embodiments, the group of drugs comprises small molecule compounds and/or ligands targeting a common signaling molecule and/or reaction implicated in the disease. In some embodiments, the drug is chosen from a group of kinase inhibitors. In some embodiments, the group of drugs comprises EGFR inhibitors, VEGF inhibitors, Ras/Raf inhibitors, Akt inhibitors, MEK inhibitors, or PDGF inhibitors. In some embodiments, the drug is chosen from the group of Lapatinib, Gefitinib, and Erlotinib. In some embodiments, the drug further affects an additional signaling pathway molecule and/or reaction.

In some embodiments, the reference topology is derived from or representative of non-diseased cells of the same cell type or tissue of origin as the cells obtained from the subject. In some embodiments, the reference topology is derived from or representative of non-diseased cells obtained from the same subject as the diseased cells. For example, in a subject diagnosed with a breast cancer, the reference topology may, in some embodiments, be derived from or be representative of cells derived from healthy breast tissue, for example, from healthy breast tissue adjacent to the breast cancer tissue. In some embodiments, for example, in embodiments, where the diseased cells are tumor cells, and where the tissue of origin of the tumor cells is known, the reference topology may be derived from or be representative of healthy cells derived from the same tissue or cells derived from healthy tissue of the same type. For example, if the subject is diagnosed with a lung cancer the tissue of origin is liver, the reference topology may, in some embodiments, be derived from or representative of a population of healthy liver cells, or of cells derived from healthy liver tissues, either obtained from the same or from another subject or other subjects.

The function and advantage of these and other embodiments of the present invention will be more fully understood from the example section below. The following example section is intended to illustrate the benefits of the present invention and to describe particular embodiments, but does not exemplify the full scope of the invention. Accordingly, it will be understood that the example section is not meant to limit the scope of the invention.

### Example

### Experimental Procedure: Phosphoprotein Dataset

HepG2 cells were purchased from ATCC (Manassas, Va.), and seeded on 96-well plates coated with collagen type I-coated (BD Biosciences, Franklin Lakes, N.J.) at 30,000 cells/well in DME medium containing 10% Fetal Bovine Serum (FBS). The following morning, cells were starved for 4 hours and treated with inhibitors and/or drugs. Kinase inhibitors were used at concentrations sufficient to inhibit at least 95% the phosphorylation of the nominal target as determined by dose-response assays (presented in [17]). AKT was chosen as the nominal target for Lapatinib, Erlotinib, and Gefitinib. The following saturated concentrations were used: p38 (PHA818637, 20 nM), MEK (PD325901, 100 nM) and cMET (JNJ38877605, 1 μM), PI3K (PI-103, 10 μM), Lapatinib at 3 uM [47], Erlotinib at 1 uM [47], Gefitinib at 3 uM [47], and Sorafenib at 3 uM (based on its inhibitory activity on ERK1/2 phosphorylation [33]). Following incubation for 45 minutes with inhibitors and/or drugs cells were treated with saturated levels of 5 ligands: Tumor Necrosis Factor alpha (TNFα) at 100 ng/ml, Interleukin 1 alpha (IL1α) at 10 ng/ml, Insulin (INS) at 2 uM, Transforming Growth Factor (TGFα) at 100 ng/ml, and Hepatocytes Growth Factor (HGF) at 100 ng/ml. Each ligand was added alone or in pairs and cell lysates were collected at 0, 5, and 25 minutes following the cytokine stimulation. The 5 and 25 minutes lysates were mixed together in 1:1 ratio and the mixed lysate was measured as an indicator of the “average early signaling response”. The 5 and 25 minute time points were identified in a preliminary experiment as the optimal time points that maximally captured early phosphorylation activities [17].

A major improvement in the present dataset as compared to [17] was the “in-vitro” averaging of the signals from 5 and 25 minutes rather than “in-silico” averaging (i.e., first both time points are measured, then we take the average). Three are the main advantages using such approach: 1) two signals are used instead of one and thus very early signaling responses can be captured, 2) the experimental cost is reduced by 50% (or more for averaging multiple time points), and 3) we achieved the averaging of some signals that could not be measured independently because their “active” state is reaching the saturation limits of our measuring instrument.

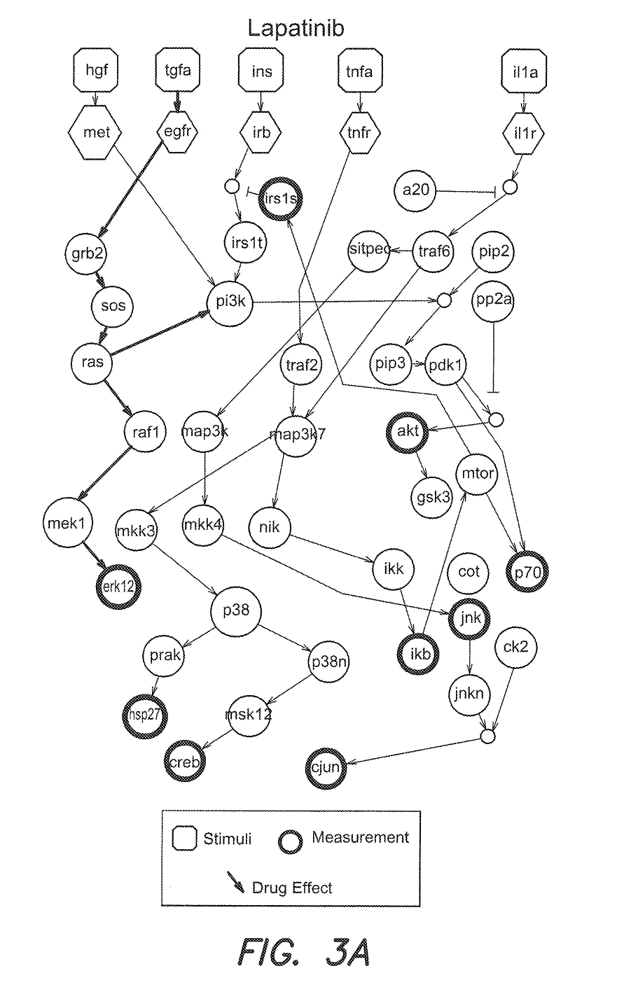

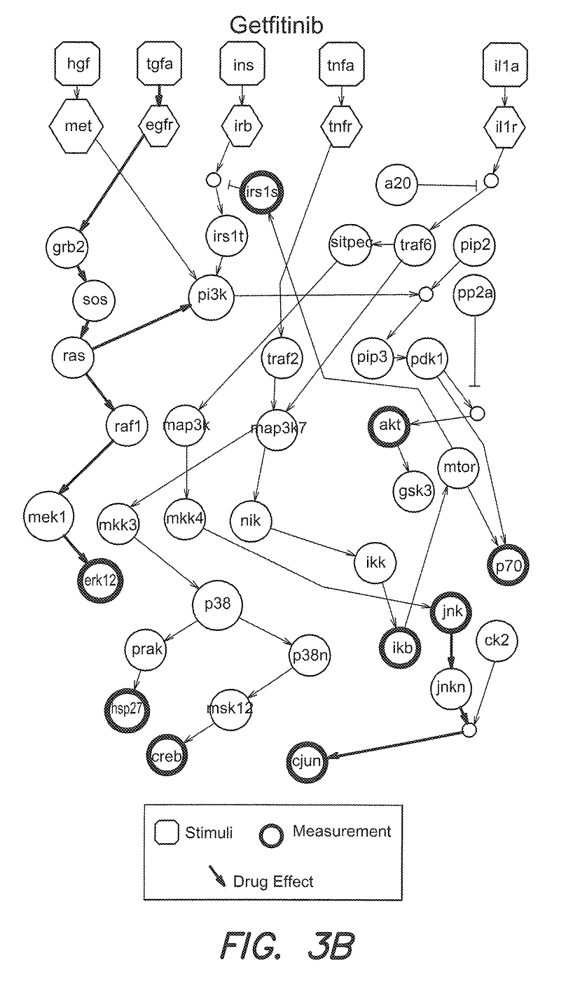

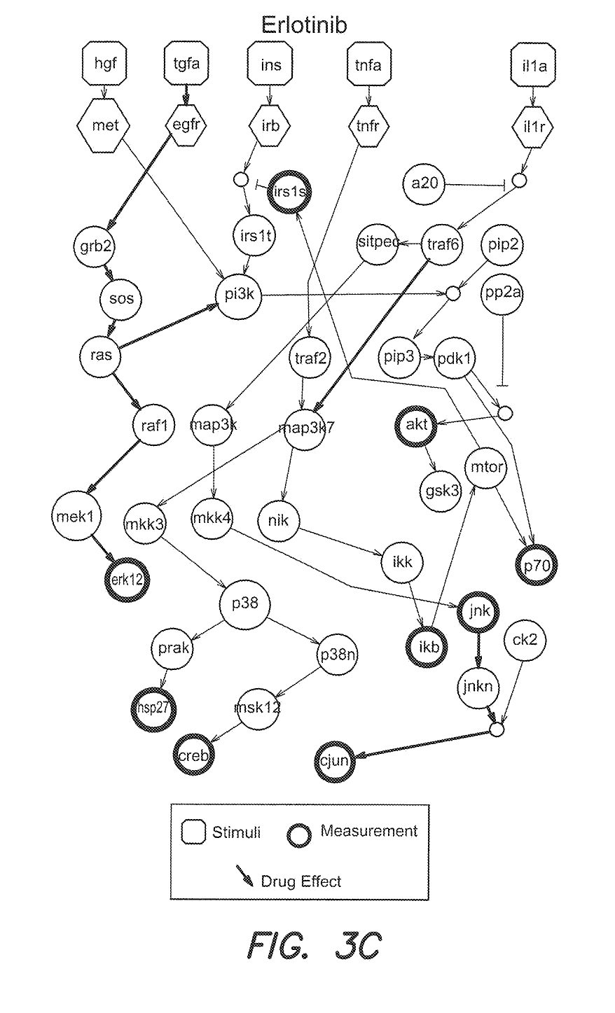

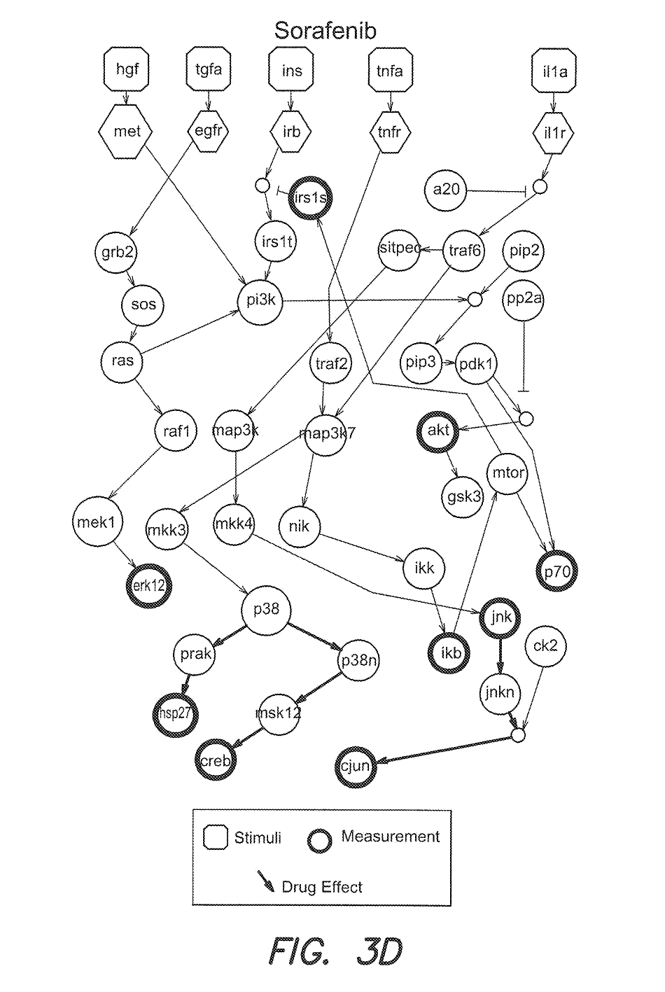

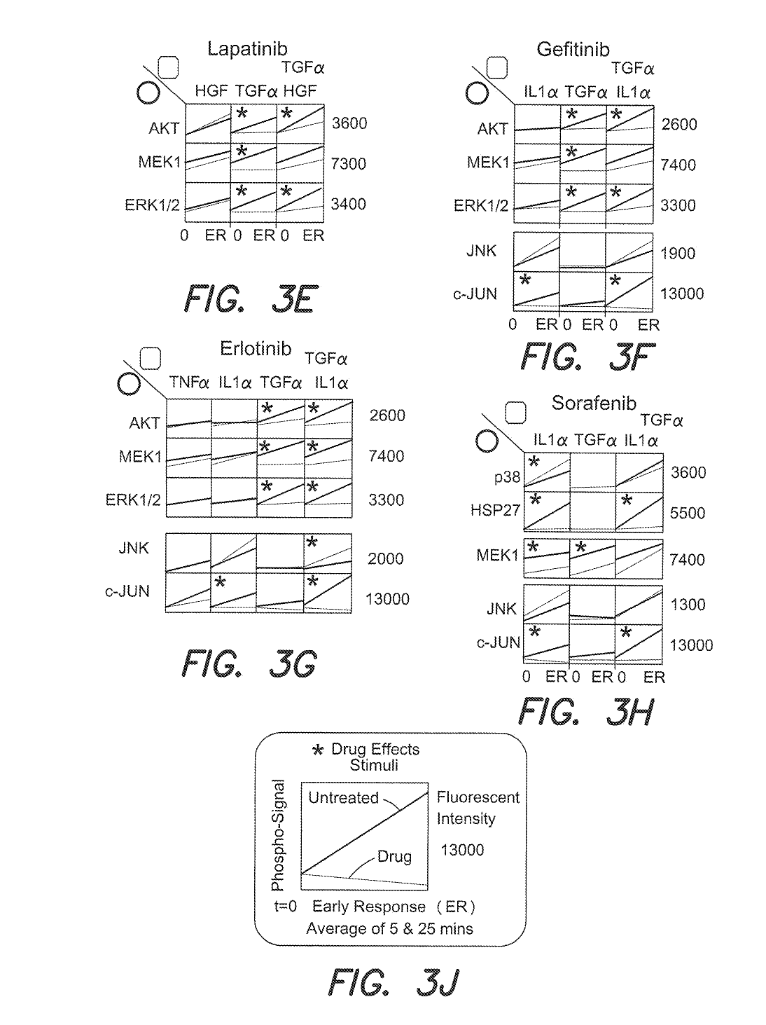

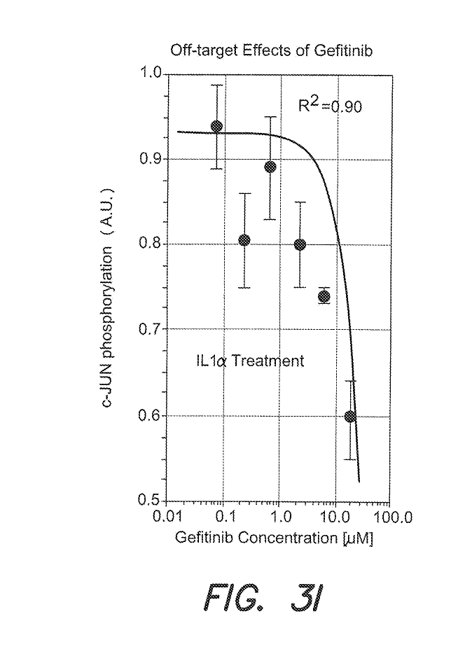

From each lysate we measured 13 phosphorylation activities that we considered “key phosphorylation events” using a Luminex 200 system (Luminex Corp, Austin, Tex.). The 13-plex phospho-protein bead set from Bio-Rad was used to assay p70S6K (Thr421/Ser424), CREB (Ser133), p38 (Thr180/Tyr182), MEK1 (Ser217/Ser221), JNK (Thr183/Tyr185), HSP27 (Ser78), ERK1/2 (Thr202/Tyr204, Thr185/Tyr187), c-JUN (Ser63), IRS-1 (Ser636/Ser639), IκB-α (Ser32/Ser36), Histone H3 (Ser10), Akt (Ser473), and IR-β (Tyr1146). Data were normalized and plotted using with DataRail [48]. For the construction of the dose response curve in FIG. 3i, HepG2 were starved for 4 hours and then incubated with Gefitinib (from 20 uM down to 27 nM-3 fold dilution) for 45 minutes followed by incubation with IL1α at 10 ng/ml final concentration for 30 minutes. Duplicate lysates were analyzed using the c-JUN (Ser63) beads in the Luminex 200 system.

### Computational Procedure: ILP Formulation

Here, we describe how the Boolean model described in [18] can be reformulated as an ILP. Note that such a transformation was recently performed for a different problem, namely the satisfiability, by [49]. A pathway is defined as a set of reactions i=1, . . . , nᵣ and species j=1, . . . , nₛ. Each reaction has three corresponding index sets, namely the index set of signaling molecules Rᵢ, inhibitors Iᵢ, and “products” Pᵢ (“product” can also correspond to the phosphorylation level of the protein). These sets are all subsets of the species index set (Rᵢ,Iᵢ,Pᵢ⊂{1, . . . , nₛ}). Typically, these subsets have very small cardinality (few species), e.g., |Rᵢ|=0,1,2;|Iᵢ|=0,1;|Pᵢ|=1,2;|Rᵢ|+|Iᵢ|=1,2. A reaction takes place if and only if all reagents and no inhibitors are present. If a reaction takes place, all products are formed. Note that reactions without products as well as reactions with neither reagents nor inhibitors will be excluded here.

While typically the set of species is known, the set of reactions is not known. Rather, only a superset of potential reactions is postulated. The goal of the proposed formulation is to find an optimal (in some sense) set of reactions out of such a superset. To that extent binary variables yᵢ are introduced, indicating if a reaction is possible or not (yᵢ=0 connection not present, yᵢ=1 connection present).

A set of experiments is performed, indexed by the superscript k=1, . . . , nₑ. In each experiment a subset of species is introduced to the system and another subset is excluded from the system. These are summarized by the index sets M^{k,1} and M^{k,0} respectively (two for each experiment). In the proposed formulation, constants are introduced for all such species, respectively xⱼ^{k}=1 and xⱼ^{k}=0. In the following it will be assumed that these species do not appear as products in any reaction; this assumption is not limiting, since in the experiments performed only extracellular species and inhibitors are manipulated. In the experiments a third subset of the species is measured (index set M^{k,2}) and for the remaining species no information is available. In the proposed formulation for each of the experiments and each such species a binary decision variable xⱼ^{k}ε{0,1} is introduced indicating if the species j is present (xⱼ^{k}=1) or not (xⱼ^{k}=0) in the experiment k according to the model predictions. It is proved that in the absence of loops, xⱼ^{k}ε[0,1] can be used for species that are not input species (see below for proof). This has some computational advantages.

The last group of variables zᵢ^{k} introduced indicate if reaction i will take place (zᵢ^{k}=1) or not (zᵢ^{k}=0) in the experiment k according to the model predictions. It is proved that a real variable zᵢ^{k}ε[0,1] can be used equivalently (see below for proof). This reformulation has somel computational advantages.

For the case that a species is measured, the measurement is defined as xⱼ^{k,m}. For Boolean measurements xⱼ^{k,m}ε{0,1}; otherwise xⱼ^{k,m}ε[0,1] (assuming a scaling as aforementioned). The primary objective function is formed aiming to minimize the weighted error between model predictions and measurements Σ_{j,k}αⱼ^{k}|xⱼ^{k}−xⱼ^{k,m}|. The absolute value is reformulated as xⱼ^{k,m}+(1−2xⱼ^{k,m})xⱼ^{k}. It can be easily verified that for binary xⱼ^{k} and for xⱼ^{k,m}ε{0,1} this reformulation is valid:

1. xⱼ^{k}=0;

xⱼ^{k,m}+(1−2xⱼ^{k,m})xⱼ^{k}=xⱼ^{k,m}+(1−2xⱼ^{k,m})0=xⱼ^{k,m}=|xⱼ^{k,m}|=|xⱼ^{k}−xⱼ^{k,m}|.

2. xⱼ^{k}=1;

xⱼ^{k,m}+(1−2xⱼ^{k,m})xⱼ^{k}=xⱼ^{k,m}+(1−2xⱼ^{k,m})1=1−xⱼ^{k,m}=|xⱼ^{k,m}|=|xⱼ^{k}−xⱼ^{k,m}|.

Note also that alternative norms, such as least-squares errors, could be also used. The resulting optimization problem would still be an ILP, since the objective function involves only integer variables. For instance for the least-square error objective function the following linear reformulation is valid:

(xⱼ^{k}−xⱼ^{k,m})²=(xⱼ^{k})²−(2xⱼ^{k}xⱼ^{k,m})+(xⱼ^{k,m})²=(xⱼ^{k})−(2xⱼ^{k}xⱼ^{k,m})+(xⱼ^{k,m})²

The secondary objective is to minimize the weighted number of possible reactions Σᵢβᵢyᵢ. In multiobjective optimization typically the concept of Pareto-optimal or noninferior solution is introduced, i.e., a set of decision variable values, such that if one tries to improve one objective, another will be degraded [50]. The set of Pareto points forms the Pareto-optimal curve. Here, however, the primary objective is considered much more important than the secondary objective. Therefore, a single Pareto-optimal point is obtained, by first minimizing the primary objective and then the secondary objective by requiring that the former (more important) objectives are not worsened, see also [51]-[53].

The ILP proposed can be summarized as:

$$\begin{matrix} {min}_{X , y , Z} {\sum }_{k = 1}^{{n}_{e}} {\sum }_{j ∈ {M}^{k , 2}} {α}_{j}^{k} ( {x}_{j}^{k , m} + ( 1 - 2 {x}_{j}^{k , m} ) {x}_{j}^{k} ) ; {\sum }_{i = 1}^{{n}_{r}} {β}_{i} {y}_{i} & ( 1 ) \\ s . t . {\sum }_{i = 1}^{{n}_{r}} {a}_{i}^{l} {y}_{i} \leq {b}^{l} , l = 1 , … , {n}_{c} , & ( 2 ) \\ {z}_{i}^{k} \leq {y}_{i} , i = 1 , … , {n}_{r} , k = 1 , … , {n}_{e} . & ( 3 ) \\ {z}_{i}^{k} \leq {x}_{j}^{k} , i = 1 , … , {n}_{r} , k = 1 , … , {n}_{e} , j ∈ {R}_{i} & ( 4 ) \\ {z}_{i}^{k} \leq 1 - {x}_{j}^{k} , i = 1 , … , {n}_{r} , k = 1 , … , {n}_{e} , j ∈ {I}_{i} & ( 5 ) \\ {z}_{i}^{k} \geq {y}_{i} + {\sum }_{j ∈ {R}_{i}} ( {x}_{j}^{k} - 1 ) - {\sum }_{j ∈ {I}_{i}} ( {x}_{j}^{k} ) , i = 1 , … , {n}_{r} , k = 1 , … , {n}_{e} . & ( 6 ) \\ {x}_{j}^{k} \geq {z}_{i}^{k} , i = 1 , … , {n}_{r} , k = 1 , … , {n}_{e} , j ∈ {P}_{i} . & ( 7 ) \\ {x}_{j}^{k} \leq {\sum }_{i = 1 , … , {n}_{r} ∷ j ∈ {P}_{i}} {z}_{i}^{k} , j = 1 , … , {n}_{s} , k = 1 , … , {n}_{e} . & ( 8 ) \\ {x}_{j}^{k} = 0 , k = 1 , … , {n}_{e} , j ∈ {M}^{k , 0} & ( 9 ) \\ {x}_{j}^{k} = 1 , k = 1 , … , {n}_{e} , j ∈ {M}^{k , 1} & ( 10 ) \\ X ∈ {\{ 0 , 1 \}}^{{n}_{e} \times {n}_{s}} , y ∈ {\{ 0 , 1 \}}^{{n}_{r}} , Z ∈ {\{ 0 , 1 \}}^{{n}_{e} \times {n}_{r}} , & ( 11 )\end{matrix}$$

where the objectives are separated by a semi-colon. Note that for the elements of the matrices) X and Z, the row index (experiment) is indicated as superscript, and the column index (species and reactions respectively) is indicated as subscript.

In formulation (1)-(11) for the manipulated species binary decision variables along with the constraints (9) and (10) are introduced. This simplifies notation. In the implementation, these variables are replaced by constants. Alternatively the preprocessor of the optimization solver can be used to exclude these trivial variables.

In the following the reasoning for the formulation is given. The first set of constraints, i.e., (2) allow the modeler to limit the combinations of connectivities considered. For instance, suppose that two reagents R1, R2 form a product P, but it is not known if both reagents (AND) or either (OR) are required. This can be modeled as three potential reactions

R1+R2→P r1

R1→P r2

R2→P, r3

with the additional constraint that excludes r2 and r3, which can be modeled as two linear inequalities:

yᵣ_{₁}+yᵣ_{₂}≦1

yᵣ_{₁}+yᵣ_{₃}≦1

The constraints (3) indicate that a reaction can only take place if it is possible (yᵢ=1). This can be seen easily, since yᵢ=0, gives zᵢ^{k}≦0 and together with zᵢ^{k}ε{0, 1} we obtain zᵢ^{k}=. Similarly, the constraints (4) and (5) ensure respectively that a reaction can only take place if all reagents and no inhibitors are present. If for instance a reagent is absent, zᵢ^{k}=0 is enforced, and the other constraints are redundant. On the other hand, the constraints (6) enforce that if a reaction is possible, all reagents are present, and no inhibitors are present, then the reaction will take place (zᵢ^{k}=1).

The constraints (7) ensure that a species will be formed if some reaction in which it is a product occurs. Note that multiple reactions can give the same species; mathematically this will result in redundant constraints. In contrast, the constraints (8) enforce that a species will not be present if all reactions in which it appears as a product do not occur. Recall that manipulated species are not considered as products in reactions. Note also, that it would be possible to combine the constraints (7) into a single constraint for each species, e.g.,

$${x}_{j}^{k} \geq {\sum }_{i = 1 , … , {n}_{r} ∷ j ∈ {P}_{i}} {z}_{i}^{k} / {\sum }_{i = 1 , … , {n}_{r} ∷ j ∈ {P}_{i}} 1 , j = 1 , … , {n}_{s} , k = 1 , … , {n}_{e} ,$$

but this would result in weaker LP-relaxations. Also the reformulation of xⱼ^{k} to [0,1] would no longer be exact.

In the present study, our ILP formulation was utilized in two different circumstances. For the creation of the cell-type specific pathway using combinations of inhibitors and stimuli our ILP formulation included 27887 constraints and 9732 variables. For each drug case, where the reduced and optimized pathway was utilized, we had 2477 constraints and 947 variables.

### Computational Procedure: Goodness of Fit

For the goodness of fit, we calculated the percentage error as:

$$Error = {\sum }_{j = 1}^{{n}_{s}} \vert {x}_{j}^{k , m} - {x}_{j}^{k} \vert / {n}_{s , m} \cdot 100 \%$$

Note that for binary xⱼ^{k} and xⱼ^{k,m}ε[0,1] the percentage error cannot be 0% even when there is no mismatch between model and experiment data. Another way to quantify the goodness of fit is by counting the number of mismatches: the cases where the rounded experimental value (0 or 1) is not the same with the computational value, or in other words, when experimental—computational error is more than 0.5.

### Equivalent Reformulation as MILP

The ILP proposed is given below and described in more detail elsewhere herein:

$$\begin{matrix}{min}_{X , y , Z} {\sum }_{k = 1}^{{n}_{e}} {\sum }_{j ∈ {M}^{k , 2}} {α}_{j}^{k} ( {x}_{j}^{k , m} + ( 1 - 2 {x}_{j}^{k , m} ) {x}_{j}^{k} ) ; {\sum }_{i = 1}^{{n}_{r}} {β}_{i} {y}_{i} & ( 1 ) \\ s . t . {\sum }_{i = 1}^{{n}_{r}} {a}_{i}^{l} {y}_{i} \leq {b}^{l} , l = 1 , K , {n}_{c} , & ( 2 ) \\ {z}_{i}^{k} \leq {y}_{i} , i = 1 , K , {n}_{r} , k = 1 , K , {n}_{e} . & ( 3 ) \\ {z}_{i}^{k} \leq {x}_{j}^{k} , i = 1 , K , {n}_{r} , k = 1 , K , {n}_{e} , j ∈ {R}_{i} & ( 4 ) \\ {z}_{i}^{k} \leq 1 - {x}_{j}^{k} , i = 1 , K , {n}_{r} , k = 1 , K , {n}_{e} , j ∈ {I}_{i} & ( 5 ) \\ {z}_{i}^{k} \geq {y}_{i} + {\sum }_{j ∈ {R}_{i}} ( {x}_{j}^{k} - 1 ) - {\sum }_{j ∈ {I}_{i}} ( {x}_{j}^{k} ) , i = 1 , K , {n}_{r} , k = 1 , K , {n}_{e} . & ( 6 ) \\ {x}_{j}^{k} \geq {z}_{i}^{k} , i = 1 , K , {n}_{r} , k = 1 , K , {n}_{e} , j ∈ {P}_{i} . & ( 7 ) \\ {x}_{j}^{k} \leq {\sum }_{i = 1 , K , {n}_{r} ∷ j ∈ {P}_{i}} {z}_{i}^{k} , j = 1 , K , {n}_{s} , k = 1 , K , {n}_{e} . & ( 8 ) \\ {x}_{j}^{k} = 0 , k = 1 , K , {n}_{e} , j ∈ {M}^{k , 0} & ( 9 ) \\ {x}_{j}^{k} = 1 , k = 1 , K , {n}_{e} , j ∈ {M}^{k , 1} & ( 10 ) \\ X ∈ {\{ 0 , 1 \}}^{{n}_{e} \times {n}_{s}} , y ∈ {\{ 0 , 1 \}}^{{n}_{r}} , Z ∈ {\{ 0 , 1 \}}^{{n}_{e} \times {n}_{r}} , & ( 11 )\end{matrix}$$

### Relaxation of Z

We will argue that relaxing the Z variables from binary to continuous gives an exact reformulation. It suffices to show that constraints (3)-(8) together with Xε{0,1}ⁿ^{ₑ}^{×n}^{ₛ} and yε{0,1}ⁿ^{ᵣ} imply Zε{0,1}ⁿ^{ₑ}^{×n}^{ᵣ}.

Theorem 1 Replacing Zε{0,1}ⁿ^{ₑ}^{×n}^{ᵣ} by Zε[0,1]ⁿ^{ₑ}^{×n}^{ᵣ} is an exact reformulation, in the sense that any feasible point in the new program is also feasible in the original program.

Note that Theorem 1 is a special case of Theorem 2. Nevertheless it is given separately, because it does not require the assumption of acyclical graphs. Moreover, its proof is much simpler, and is used in the proof of Theorem 2.

Proof. Take any Xε{0,1}ⁿ^{ₑ}^{×n}^{ₛ}, yε{0,1}ⁿ^{ᵣ} and Zε[0,1]ⁿ^{ₑ}^{×n}^{ᵣ} that satisfies the constraints of (1)-(11). Take any i=1,K,nᵣ and any k=1,K,nₑ. We consider two cases depending on the value of yᵢ^{k}.

1. yᵢ^{k}=0. From (3) we directly obtain zᵢ^{k}≦0 and therefore zᵢ^{k}=0.

2. yᵢ^{k}=1. We consider two subcases:

If for some jεR, we have xⱼ^{k}=0 (a reagent is missing), then zᵢ^{k}≦0 from (4) and therefore zᵢ^{k}=0. Similarly, if for some jεIᵢ we have xⱼ^{k}=1 (an inhibitor is present), then from (5) zᵢ^{k}≦0 and therefore zᵢ^{k}=0.

If for all jεR, we have xⱼ^{k}=1 (all reagents present) and for all jεIᵢ we have xⱼ^{k}=0 (all inhibitors absent), then from (6) we obtain zᵢ^{k}≧1 and therefore zᵢ^{k}=1.

Since the choice of i and k was arbitrary we have shown Zε{0,1}ⁿ^{ₑ}^{×n}^{ᵣ}.

Relaxation of Non-Input xᵢ^{k}

For the case that no loops are present in the pathway, we will argue that we can also use xⱼ^{k}ε[0,1] for all species but the input species. In typical pathways the majority of species are noninput species. The formal definition of input species is

Definition 1 (Input species) Species j that are not products in any reaction, i.e., T≡{jε{1,K,nₛ}:j∉∪ᵢ₌₁ⁿ^{ᵣ}Pᵢ} are termed input species.

Theorem 2 Suppose that the pathway proposed contains no loops. In (1)-(11) replacing Zε{0,1}ⁿ^{ₑ}^{×n}^{ᵣ} by Zε[0,1]ⁿ^{ₑ}^{×n}^{ᵣ} and xⱼ^{k}ε{0,1} by xⱼ^{k}ε[0,1] for all j∉T (for all non-input species) is an exact reformulation, in the sense that any feasible point in the new program is also feasible in the original program.

Note that input species cannot be relaxed, for otherwise z, E {0,1} would not be ensured. The proof idea is that because the potential pathway form a directed graph, we can proceed from the “top” to the “bottom”. In doing so we establish that both xⱼ^{k} and zᵢ^{k} are forced to be integer.

Proof Take any Xε[0,1]ⁿ^{ₑ}^{×n}^{ₛ}, yε{0,1}ⁿ^{ᵣ} and Zε[0,1]ⁿ^{ₑ}^{×n}^{ᵣ} that satisfies the constraints of (1)-(11) and that also satisfies xⱼ^{k}ε{0,1}, for all jεT (all input species are binary).

In the proof of Theorem 2 we have established that if for a given reaction i and experiment k, we have xⱼ^{k}ε{0,1} for all jεRᵢ∪Iᵢ (all reagents and inhibitors are binary), then we also obtain zᵢ^{k}ε{0,1}.

Take kε{1,K,nₑ} (an arbitrary experiment) and jε{1,K,nₛ} (an arbitrary species). We will argue that if zᵢ^{k}ε{0,1} for all iε{1,K,nᵣ}:jεPᵢ (for all reactions for which the species is a product) then yⱼ^{k}ε{0,1}. There are essentially two cases:

1. If for some iε{1,K,nᵣ}:jεPᵢ we have zᵢ^{k}=1 then by (7) we obtain xⱼ^{k}≧1 and therefore xⱼ^{k}=1.

2. If for all iε{1,K,nᵣ}:jεPᵢ we have zᵢ^{k}=0 then by (8) we obtain xⱼ^{k}≦0 and therefore xⱼ^{k}=0.

It is clear that in the absence of loops the above two arguments propagate through the pathway. From an arbitrary species jε{1,K,nₛ} we can traverse the graph in reverse direction and reach the input species in a finite number of steps (a reverse path). Due to the absence of loops, each species depends only on the species which are “further up” in the pathway.

### Construction of Phosphoproteomic Datasets

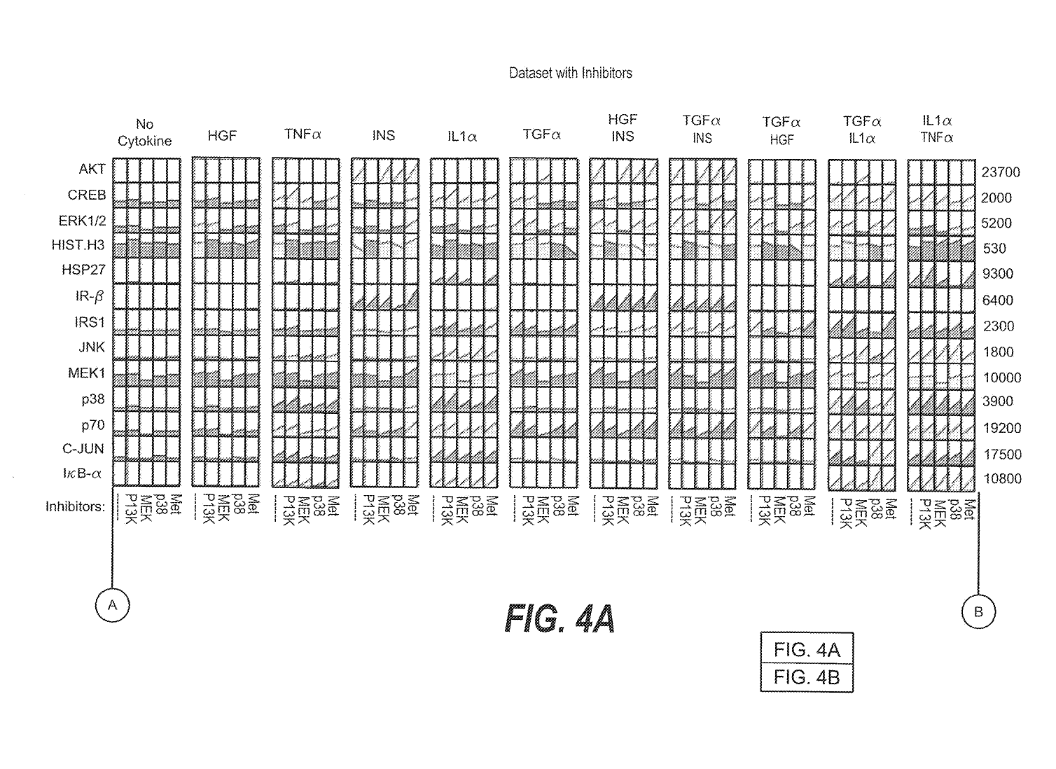

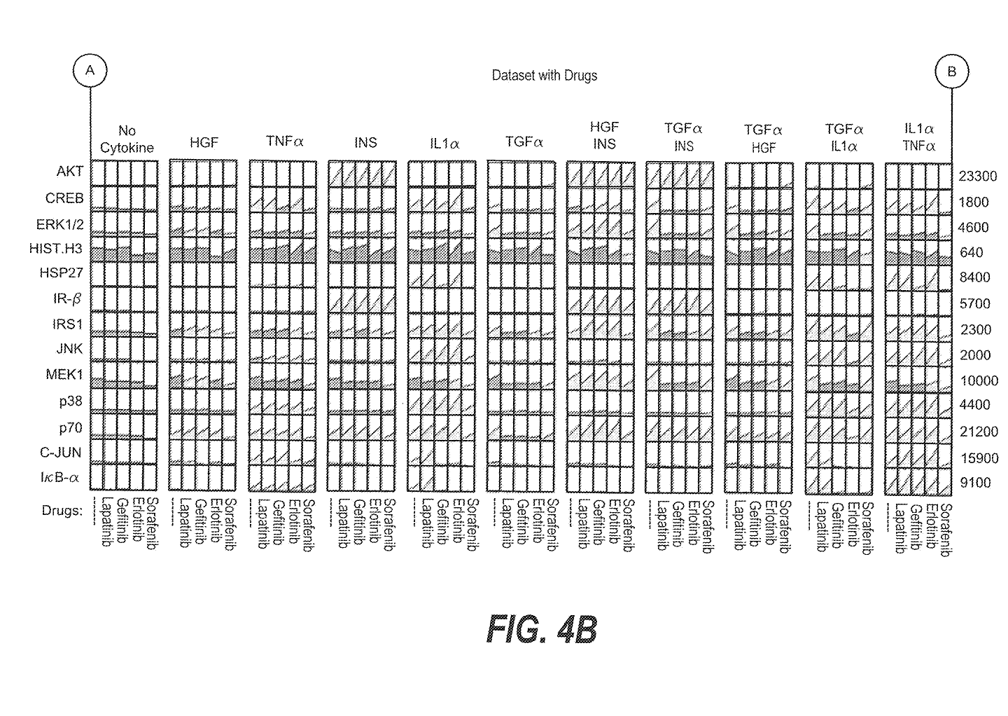

High-throughput bead-based ELISA-type experiments using xMAP technology (Luminex, Tex., USA) are performed as briefly described in the Materials and Methods section and in [17]. We create two datasets: one for the construction of cell-type specific topology and another for the identification of the mechanisms of drug actions. To do that, HepG2s are stimulated in 10 different ways with combinatorial treatments with a diverse set of 5 ligands (TNFα, IL1α, HGF, INS, TGFα, and no stimuli) and either 4 highly selective inhibitors (PI3K, MEK, p38, cMET, and no inhibitor) or 4 commercial drugs (EGFR inhibitors Lapatinib, Erlotinib and Gefitinib, and the “dirty” inhibitor Sorafenib) (FIGS. 1b and 1d). For the purpose of this paper, we refer to “inhibitors” as the compounds for which we know the target and we use them in a concentration capable to block ˜95% of the downstream protein. Conversely, we refer to “drugs” as the compounds for which we assume no a-priori knowledge of their target. For each combination of cytokine and drug/inhibitor we collect cell lysates at 5 and 25 minutes. The two time points are pooled together in 1:1 ratio and the mixed lysates are used as an indicator of the “average early signaling response”. For each treatment we measure 13 protein phosphorylations that we consider “key protein activities” (raw data in FIG. 4). The key phosphorylation signals (listed in Materials and Methods) are chosen based on the availability of the reagents and quality controls performed at the early phases of the experimental setup [17]. The raw data (arbitrary fluorescent intensities) are normalized to fit logic models as described in [18] using a non-linear transformation that converts raw data into values between 0 and 1 where 1 corresponds to the fully activated state and 0 to no-activation. It has to be noted that logic-transformed data depends on what should be considered “protein activation” (transformed value >0.5), a criterion that is embedded in the transformation function and accounts for signal-to-noise limits, saturation of the detection scheme, and eliminates biases that could have been introduced by the variability of antibody affinities [18].

### Generic Pathway Assembly and Visualization

The generic pathway map is constructed in the neighborhood of the 5 stimuli and the 13 measurements. The ubiquitous presence of conflicting reports on pathway maps and alternative protein names makes this step a highly nontrivial one. We explored several pathway databases including STKE, Pathway Interaction Database, KEGG, Pathway Commons, Ingenuity, and Pathway Studio [23],[24]. Our limited intracellular protein coverage makes impractical the reduction of very large pathway datasets such as those found in Pathway Commons. Here, we create the initial topology from the union of canonical pathways found in Ingenuity (Redwood City, Calif.) with subsequent manual curation.

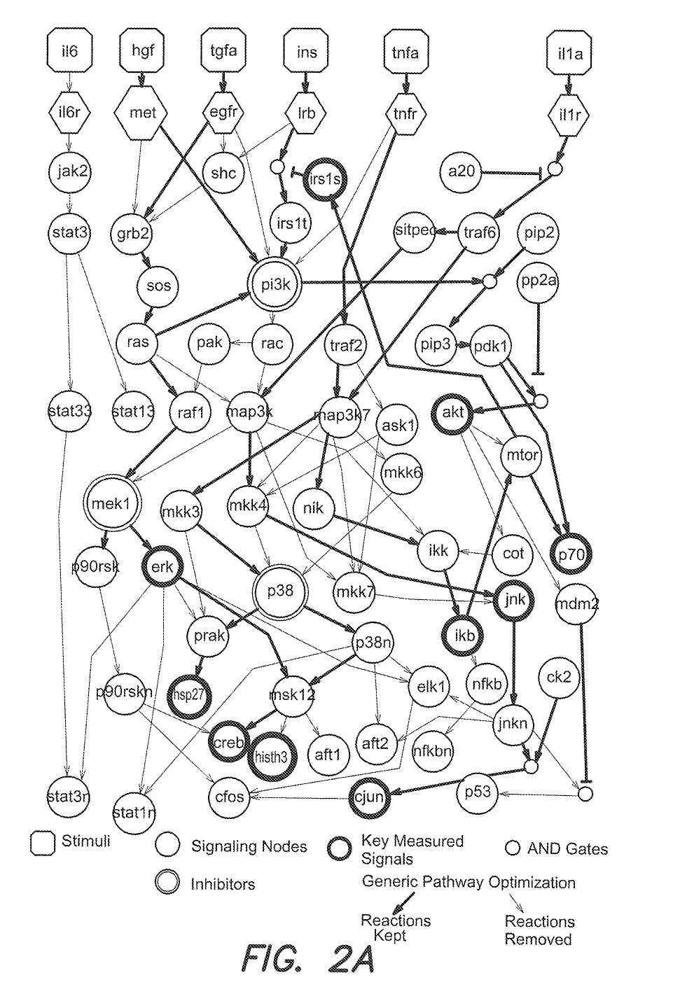

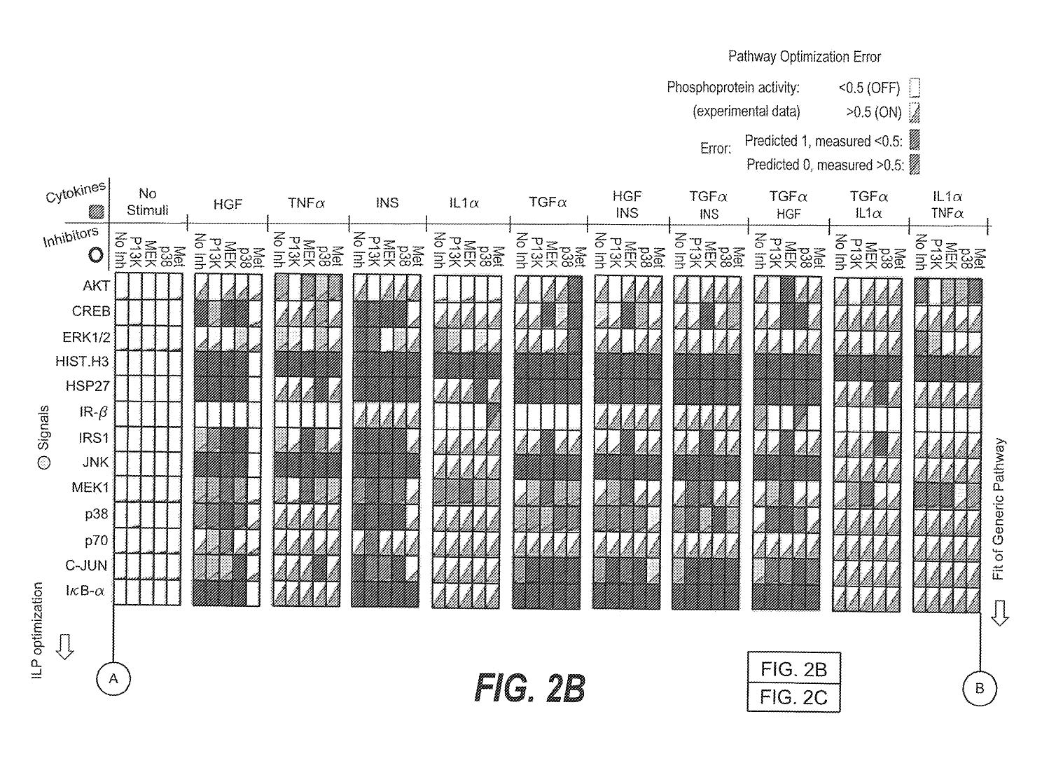

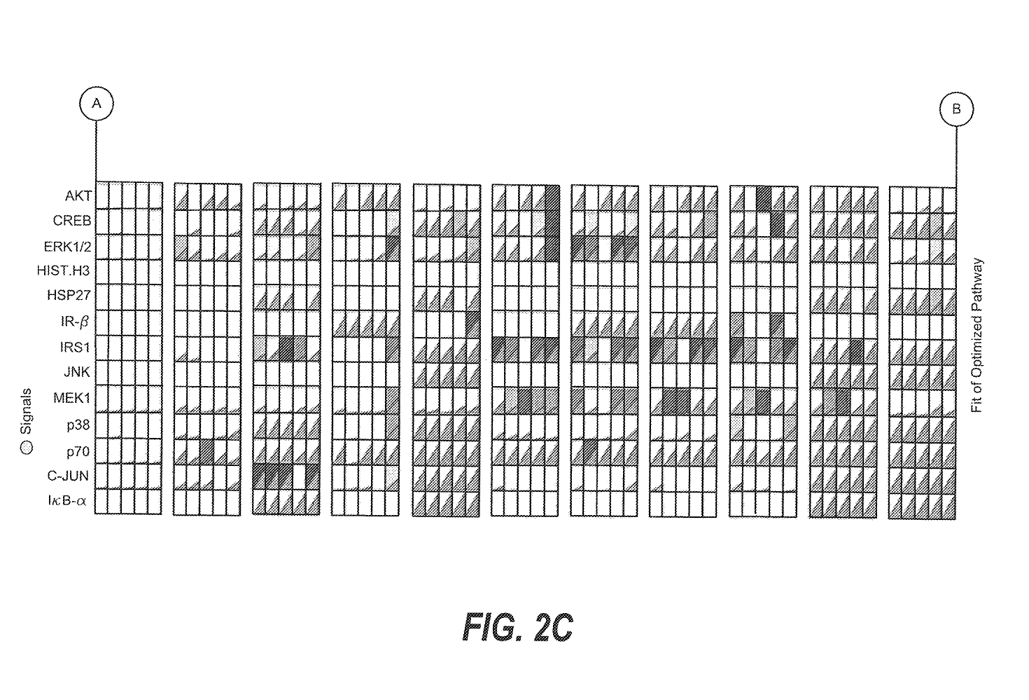

A detailed description of Boolean representation of pathways can be found elsewhere [18], [25]-[29]. In the present manuscript as opposed to [18], the connectivity in our pathway (FIG. 2, left panel) is represented with OR gates and only few connections (represented with small black circles in FIG. 2) require an AND gate. We are therefore not comparing OR vs. AND gates, but rather assuming our pathways to be ‘causal’ graphs, and since there are a few AND gates we refer to it as Boolean model.

### Construction of Cell-Type Specific Pathway Via ILP Formulation

The formulation for the optimal pathway identification is a 0-1 Integer Linear Program, i.e., an optimization problem with binary variables and linear constraints (see Materials and Methods). The optimizer picks values for the decision variables, such that the logical constraints are satisfied and the objective(s) optimized. The primary objective is to find an optimal pathway, i.e., a pathway that best describes a set of phosphoproteomic data under a given model (e.g. Boolean). A secondary objective is that the pathway is as small as possible, i.e., has as few connections as possible, such that the best-possible fit of the experiments is maintained (see Materials and Methods). It is shown that some of the binary variables can be relaxed to continuous, without changing the feasible set.

The ILP is solved with the state-of-the-art commercial code (CPLEX [30],[31]) that guarantees minimal error between experimental data and the Boolean topology. The goodness of fit (percent error as described in Materials and Methods) was decreased from 36.7% on the generic map to 8.3% on the optimized map (FIG. 2). The main source of error is the inability of TGFα to activate the IRS1_s (serine residue of IRS1) (see the shaded background on the IRS1 row at the bottom panel of FIG. 2). This is a result of the infeasibility of the generic pathway to satisfy the activation of IRS1_s in a TGFα/IL1α-dependant but HGF/INS-independent manner: TGFα activation of IRS1_s requires mTOR activation via AKT which the optimization algorithm removes to satisfy the inactivation IRS1_s by INS that shares the same path with TGFα. This example highlights the importance of multi-perturbations to better constrain the optimization formulation.

FIG. 2 shows the optimized topology of HepG2s. Our ILP formulation uses two subsequently-imposed objective functions to remove reactions that do not fit the experimental data. During the optimization of the first objective the ILP formulation (A) keeps reactions that lead to phosphorylations of the key proteins and (B) removes reactions that lead to false protein activations. An example of the first case is the Insulin (INS)-induced AKT activation that is maintained via the INS→IRb→IRS1t→PI3K→PIP3→PDK1→AKT path (see INS to AKT path in FIG. 2). An example of a removed reaction is the TNFR→PI3K reaction which is removed because there is no TNFα induced AKT activation (see TNFR→PI3K→ . . . →AKT in FIG. 2). During the optimization of the secondary objective (see Materials and Methods), several reactions with no evidence of their existence (no downstream measurements, or no stimuli) are removed. In this step, the overall goodness of fit is not improved, but the size of the topology is reduced. To illustrate this case, we add to the initial topology the receptor IL6R but the associated stimulus IL6 is not introduced on the experiments. After the secondary optimization, all downstream reactions of IL6 are removed because no data are present (see reaction arrows downstream for of IL6 in FIG. 2). Similarly, all reactions downstream of the bottom-of-the-network key proteins are removed (e.g. CJUN→CFOS reaction in FIG. 2). All those reactions might be present in reality and could have been kept if the secondary objective was not present. Here, we apply the secondary objective and follow a network trimming which removes all reactions that might be present in the cell but due to the lack of measured signals or experimental conditions cannot be verified. The resulting network is significantly smaller but contains only elements for which there are solid experimental evidence that explain the topology.

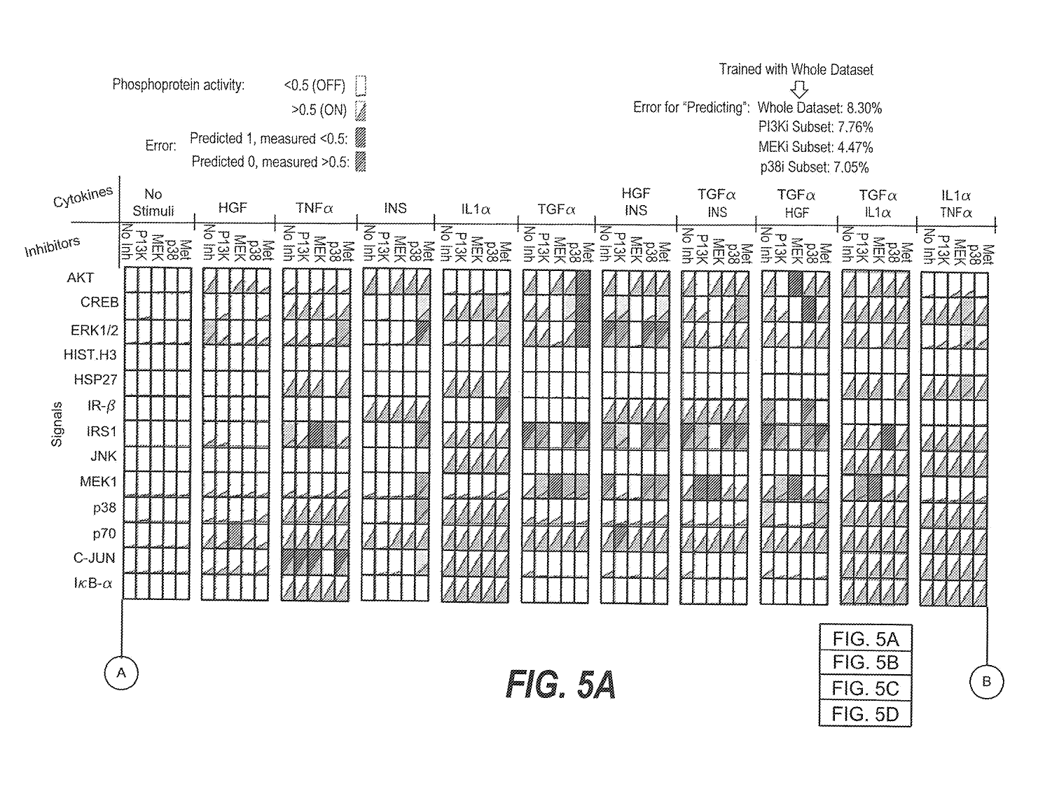

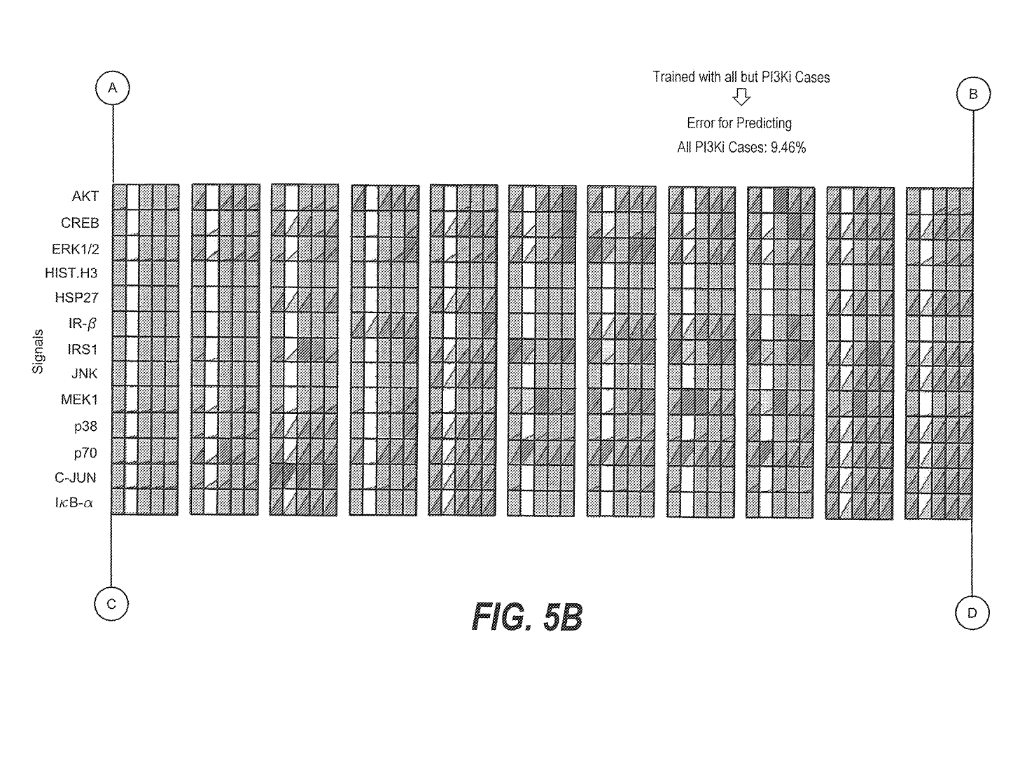

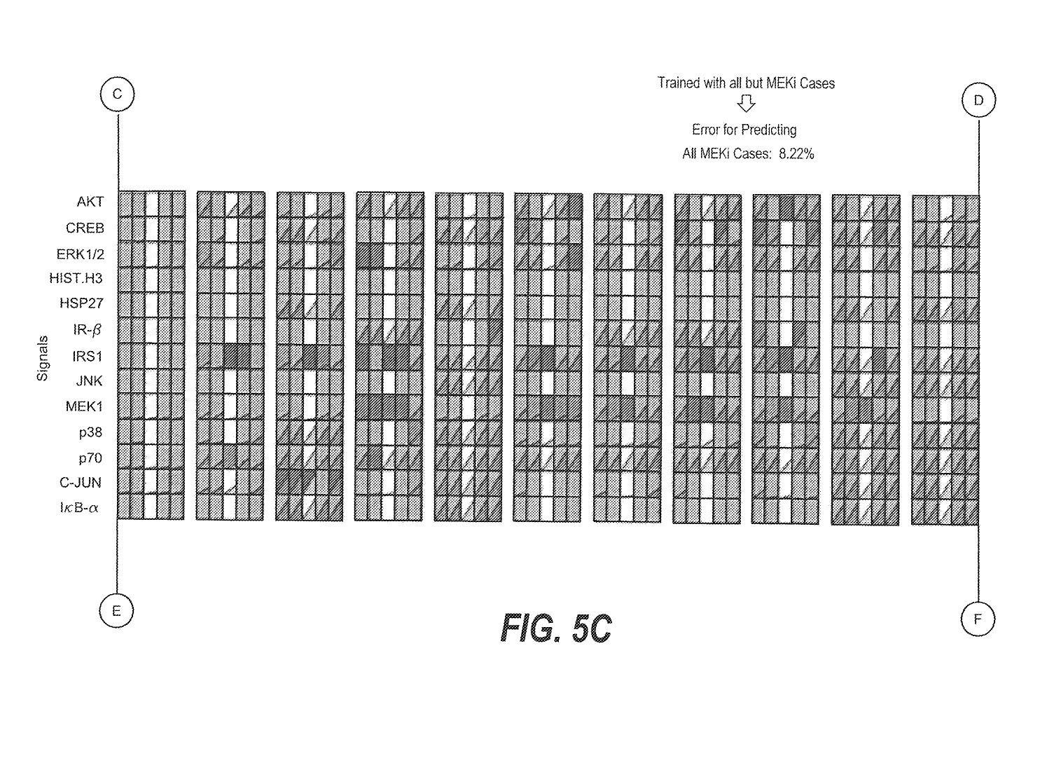

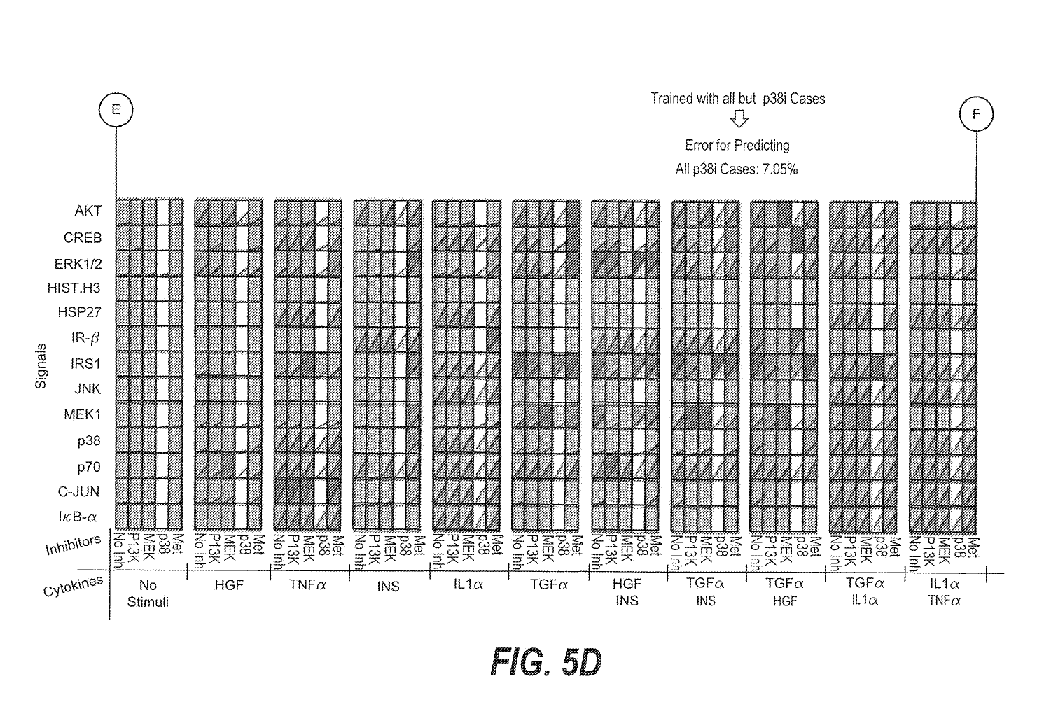

To validate our model, we also examine three scenarios where we remove 20% of our experimental data, and then we try to predict them. Specifically, we create three training datasets, each time by removing all cases where one inhibitor is present (either MEK1, PI3Ki, or p38i) and then we calculate how well our ILP-optimized map can predict each of the inhibitor cases (see FIG. 5). For the MEK1, PI3Ki, and p38i scenarios the goodness of fit is 8.22%, 9.46%, 7.05% respectively and our ILP-formulation converges on the same or slightly less optimal solutions compared to the solutions obtained when the whole dataset is used for training (4.47%, 7.76%, and 7.05% respectively)—See FIG. 5. Note that the errors given refer only to the subset considered in each case, not the entire dataset. More extensive validations for Boolean-type models on similar phospho-proteomic dataset can also be found in Saez-Rodriguez et al. [18].

Comparison with Genetic Algorithm

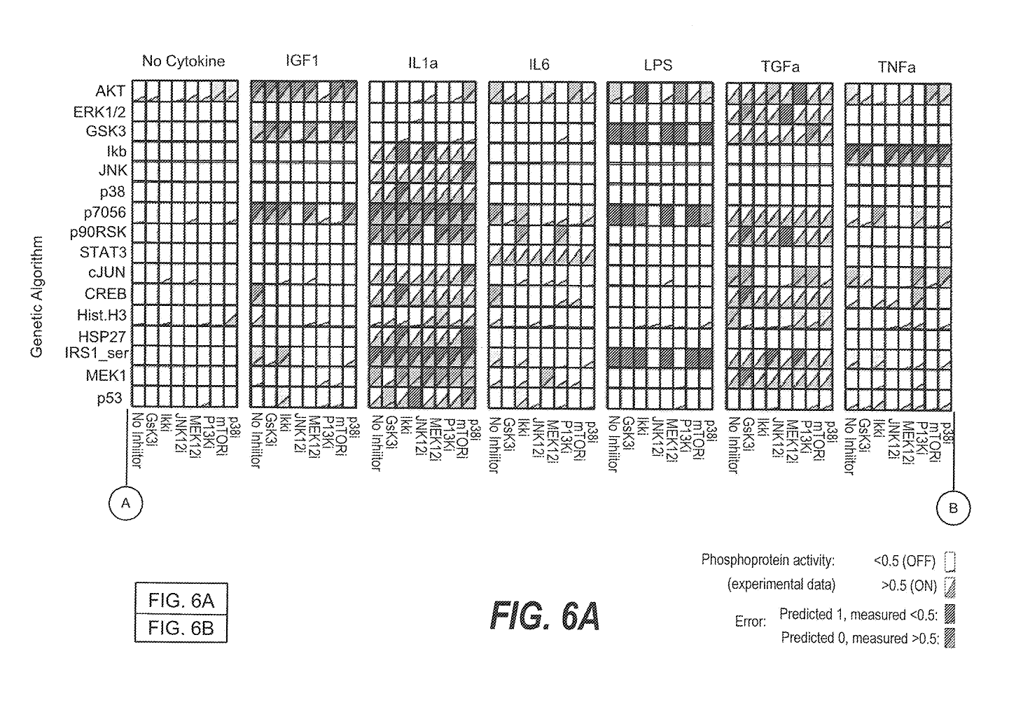

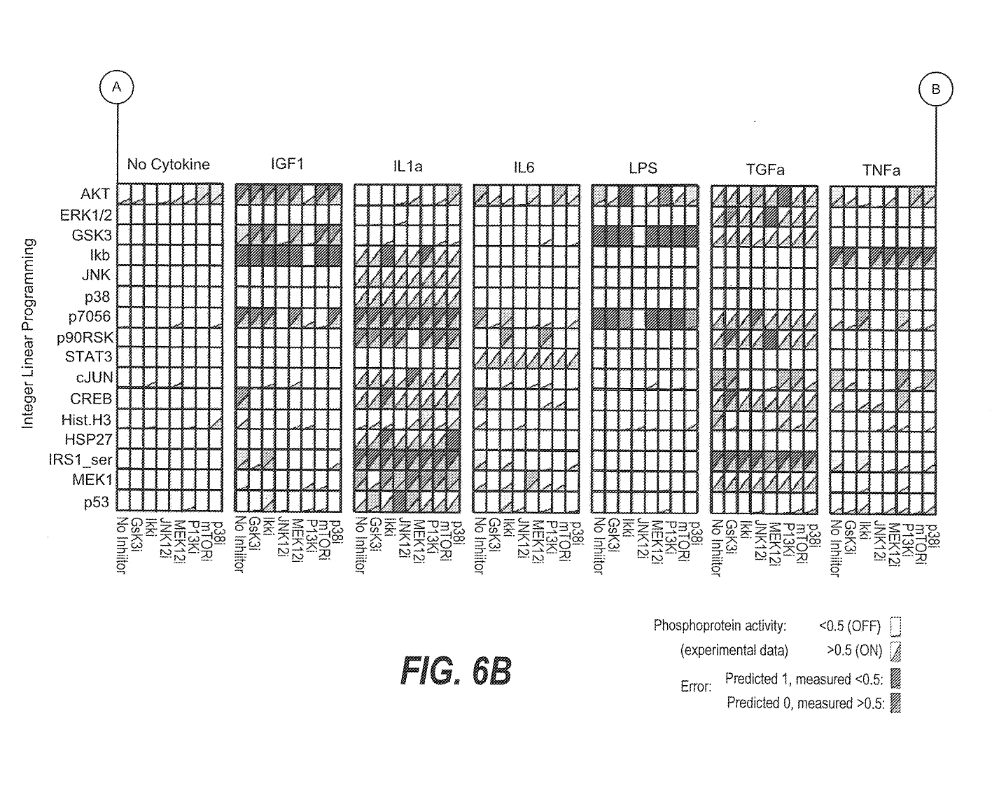

In order to compare the ILP algorithm with the previously published genetic algorithm (GA) we use the same initial topology and the same normalized dataset [18]. The two algorithms reached almost identical results (see FIG. 6). For the ILP, the computational requirements are manageable, in the order of a few seconds (14.3 seconds for this example) on an Quad Core Intel Xeon Processor E5405 (2.00 GHz, 2×6M L2,1333) running Linux 2.6.25.20 (using only one core). In comparison, the same optimization problem using GA requires approximately 1 hour on a similar power computer. The optimal pathway furnished by the ILP matches all but 98 out of 880 experimental data, as opposed to 110 mismatches in the topology furnished by the GA. It has to be noted that GA does not provide termination criteria, and it is conceivable that after even larger CPU times the GA would have achieved the same fit as the ILP. In contrast the deterministic solution of the ILP guarantees that an optimal fit (not necessarily unique) has been identified within a user-specified tolerance (10³ in our case). In addition to the guaranteed optimal solution, commercial ILP solvers are fast, robust and reliable. Note that open-source ILP solvers also exist, but in our experience are not yet adequate. Note also that for larger network topologies, the differences in CPU time will become even more dramatic, rendering the GA intractable.

The notable differences between the proposed method and the method used in [18] is mainly due to fundamental algorithmic differences: the technology behind deterministic ILP solvers (branch-and-bound, branch-and-cut) is more sophisticated than genetic algorithms, it employs the inherent linearity of the problem, and makes use of the good scalability of linear programs (sub-problems in branch-and-bound tree). In contrast, GA treats the model as a black-box and does not exploit the problem structure. Another point is that herein we used a well-established commercial solver, whereas Saez-Rodriguez et al. [18] used their own implementation of GA. Commercial deterministic ILP solvers, such as CPLEX, rely on several decades of research and development, and have extremely powerful features such as pre-processors and node selection heuristics. Thus, they typically become the default choice for ILPs.

### Identifying Drug Effects Via Drug-Induced Topology Alterations

For the identification of the drug effects we make use of the second dataset in HepG2s where drugs are applied together with the same set of ligands. In this case, the ILP formulation is being used with the HepG2 specific topology (topology obtained from the previous step) and not the generic map. We also do not impose inhibitor constrains the way we do for pathway optimization (e.g., PI3K inhibitor blocks the signal downstream of PI3K) but we let the optimization algorithm decide which reaction(s) should be removed in order to fit the drug-induced data.

The effect of Lapatinib (FIG. 3a), the most selective and specific EGFR inhibitor [32], is the complete removal of the downstream reactions of the TGFα branch: TGFα→GRB2→SOS→RAS→PI3K and RAS→RAF1→MEK1/2→ERK1/2. This resulted from the fact that Lapatinib blocks the TGFα induced MEK1/2, ERK1/2, and AKT phosphosignals (FIG. 3e). Note that the PI3K→ . . . →AKT branch is not removed because it is being used by the HGF and INS path for the activation of AKT that cannot be blocked by Lapatinib (FIG. 3e).

Gefitinib, an EGFR tyrosine kinase inhibitor, alters the topology in a very similar pattern as Lapatinib, but, interestingly enough, it also results in the removal of the JNK→c-JUN branch (FIG. 3b). Closer examination of the raw data (FIG. 3f) shows a potent inhibition of IL1α- and (IL1α+TGFα)-induced cJUN activity upon Gefitinib treatment. To follow up this interesting off-target effect, we did a dose-response experiment where Gefitinib shows that it can reduce the activation of cJUN signal induced by the IL1α stimuli (FIG. 3i). We believe that the inhibition of cJUN is not due to the binding of Gefitinib in the upstream molecule JNK but a collective effect of signaling inhibitions in several species that take part in the path between IL1α and cJUN. For this reason, a fitting with a typical dose response curve has been avoided and a simple linear equation has been used instead (FIG. 3i). Erlotinib, another EGFR inhibitor, has the same effects as Gefitinib (FIG. 3c) but at the same time shows an effect in the TRAF6→MAP3k7 reaction. This effects is probably because IκB-α is inhibited in an IL1α-dependent but TNFα-independent manner (see IκB-α signals upon IL1α and TNFα stimuli in FIG. 4); the only way for the ILP to satisfy this behavior is to remove the transmission of signal before the merging of TNFα and IL1α paths which can be done through the TRAF6→MAP3K reaction.

The “dirty” Raf inhibitor Sorafenib shows a very different profile: it also blocks the JNK→c-JUN branch (FIG. 3d) and in addition affects the p38 path (see complete HSP27 inhibition upon IL1α treatment in FIG. 3h). An interesting observation is that network optimization does not remove the RAF→ERK1/2 reaction despite the fact that RAF is the main target of Sorafenib. Close inspection of the data shows that Sorafenib reduces but does not block the MEK1 phosphorylation (see MEK phosphorylation in FIG. 3h). This is in agreement with previous published results where Sorafenib does not inhibit activation of the RAF/MEK/ERK pathway in all human tumor cell lines [33] a finding that highlights the importance of in-vivo assays for the quantification of drug effects.

### Discussion

In this article, we present an unbiased phosphoproteomic-based approach and an optimization formulation to construct cell-type specific pathways and to identify drug effects on those pathways. For the pathway construction, we track 13 key phopshorylation signals in 55 different conditions generated by the combinatorial treatment of stimuli and inhibitors. Using Integer Linear Programming (ILP) for pathway optimization we take a generic network of 74 proteins and 105 reactions and construct a cell-type specific network of 49 proteins and 44 reactions that spans between the 5 stimuli and the 13 measured phosphorylated proteins. In this network, we monitor 4 cases of drug-induced pathway alterations using a similar computational scheme.

In comparison to all other protein-based target identification approaches, our method is not based on measurements of drug affinities either by in vitro or in vivo assays. Instead, we use an “operative” signaling network and rely on key phosphorylation events and a-priori knowledge of possible connections to reveal the topology and monitor its alterations under the presence of the drug. Thus, our method is expandable to any type of intracellular perturbations such as ATP-based and allosteric inhibitors, RNAi, shRNA etc. Since no bait or MS is required, we have simple ELISA-type experimental procedure with minimal requirements of cell starting protein (30,000 cells per condition), without affinity immobilizations, protein fractionations, or carefully optimized wash conditions. With our current semi-automated procedures in our lab (robotic liquid handlers), we can achieve total experimental and computational time for a similar size experiment in less than a week. On the other side, our approach can only detect signaling alterations in topologies bounded between the applied stimuli and the measured phosphorylated proteins and it misses off-target effects outside the constructed network. The expansion of the constructed network depends primarily on three factors: highly curated generic topology, multiplex assay availability for “key” phosphorylation measurements, and experimental cost. We believe that the explosive growth of multiplexed phosphoproteomic assays, the rapid reduction of the cost per datapoint, and the significant improvement in quality of several pathways databases will significantly increase the searching space for drug effects using our proposed methodology. However, our search space will always be significantly smaller compared to whole-genome based approaches [8]-[11] because it requires (a) the input of a generic pathway which is available only in well-studied pathways and (b) good quality antibodies for the detection scheme. By merging our phosphoproteomic method with genome-wide screening techniques, we might be able to combine the strengths of both approaches and increase the searching space for off-target drug effects.

An important aspect of the current approach is the construction of pathway maps. Pathway construction is a major endeavor in biology and a variety of experimental [34]-[38] and computational approaches that span from data-driven methodologies (e.g., statistical, unsupervised machine learning) to topology-based methods (e.g., kinetic models based on ordinary differential equations-ODEs) [17], [35], [38]-[41] have been developed. Our approach, which is based on Boolean (logical) modeling [26]-[28],[42], represents a simplified topology-based method. Compared to ODE-based methods, a logic model has limited abilities to model kinetic behavior [25] (especially when modeling feedback loops in single-step logic models) or even to model the protein activity in a continuous fashion. On the flip side, logic models do not require parameter estimation (sometimes ill-defined from lack of experimental data) and thus can be applied for the simulation of large topologies. A refinement of the model formalism into multistep logic [28], fuzzy logic [43], or ODE-based logic systems [44] may provide a more precise simulation of the activity and time-dependency of the signaling network. Taking into account the current limitations of experimental assays (throughput, sensitivity, reliability, cost) we believe that Boolean modeling is the method of choice with high predictive power when large topologies are studied.

Optimizing pathway topologies is a relatively new approach for the construction of cell-type specific pathways. Using Boolean topology and Genetic Algorithm (GA) for an optimization scheme, Saez-Rodriguez et al. [18] are able to fit a generic map to cell-type specific map from phosphoprotein data. Here we present an alternative method of optimal pathway identification based on ILP. Compared to GA, our algorithm gives guaranteed globally optimized map (the solution identified is guaranteed to be no worse than 0.001 than any other possible solution). Additionally, the computational cost has cut down dramatically and allows pathway optimization with ˜70 species to be performed on a desktop computer in a matter of few seconds. Due to minimal computational requirements ILP can be used for the construction of large pathways (assuming that experimental capabilities can by matched) and for the exploration of alternative reactions beyond the generic topology to further improve the optimal fit. However, several factors should be addressed before expanding our formulation to larger topologies. Although our formulation is able to identify a globally optimal solution, additional optimal solutions might exist [18] in the same generic network and further more solutions might arise when the optimization formulation is relaxed. Larger and more interconnected networks increase the number of solutions that are equally (or near equally) optimal. A possible way to circumvent this problem is to reduce our network using techniques that have been described previously in graph theory or in [18]. Being aware of those limitations in the present manuscript we described a “simple” and not highly interconnected network in order to minimize redundancy of solutions. To address the issue of finding a both unique and optimal solution we are currently working on two complementary approaches: (a) instructing the ILP solver to furnish a pool of near-optimal solutions and (b) devising “clever stimulations” by taking into account experimental limitations (i.e., combination of inhibitors, stimuli, and key protein measurements) that maximally constrains the optimization scheme and gives smaller number of unique solutions.

When applied in HepG2s, our approach identifies both known and unanticipated results. As a positive control, it removes the TGFα branch upon EGRF drug treatments. Another easily understandable effect is Sorafenib's inhibition of the pathway downstream of p38 which can be explained by the drug's target affinity to p38α and p38β [32],[45]. A surprising effect is the removal of the JNK→cJUN reaction under the influence 3 out of 4 cancer drugs Erlotinib, Gefitinib and Sorafenib. Interestingly, kinase profiles of those drugs [32] shows no medium or high affinity for the directly upstream JNK1/2 kinases. Despite that, Gefitinib shows a significant reduction of the cJUN activity upon IL1α treatment. A possible explanation is that the signaling propagation can collectively be attenuated from the low or medium off-target inhibitions of several kinases upstream of JNK and cJUN. This also might explain the inhibition curve in FIG. 3i, where Gefitinib inhibition of cJUN activation does not follow a typical dose-response curve. In this context, sensitivity analysis in ODE-based pathway models [46] have shown that slight changes of reaction constants can have significant attenuations on protein activities several steps downstream the network and thus inhibitory curves cannot be simulated by simplified dose-response models. Our findings also highlight a unique feature of our approach: we find effects of drug's promiscuity that cannot be identified by the direct binding of the drug to the upstream target but are the result of a collective effect of drug's interactions with several upstream molecules. Bait-based analysis cannot reveal those effects since there is no binding involved between the drug and the protein.

Understanding the interplay between cell function and drug action is a major endeavor in the pharmaceutical industry. Here, we provided a methodology to construct cell type specific maps and identify drug effects on those maps. Our ILP formulation was able to build the best possible topology from a set of a-priori determined reactions and choose those, where their presence is confirmed from high throughput phosphoprotein data. Since phosphorylation events are the ultimate reporters of protein/drug function the use of high-throughput phosphoproteomic datasets gave an advantage in data quality for modeling signaling network. We believe our approach complements standard biochemical drug profiling assays and sheds new light into the discovery of possible mechanisms for drug's efficacy and toxicity.

### REFERENCES

1. Butcher EC (2005) Can cell systems biology rescue drug discovery? Nat Rev Drug Discov 4: 461-467.
2. Goldstein D M, Gray N S, Zarrinkar P P (2008) High-throughput kinase profiling as a platform for drug discovery. Nat Rev Drug Discov 7: 391-397.
3. Fabian M A, Biggs W H, Treiber D K, Atteridge C E, Azimioara M D, et al. (2005) A small molecule-kinase interaction map for clinical kinase inhibitors. Nat Biotech 23: 329-336.
4. Janes K A, Albeck J G, Peng L X, Sorger P K, Lauffenburger D A, et al. (2003) A High-throughput Quantitative Multiplex Kinase Assay for Monitoring Information Flow in Signaling Networks Application to Sepsis-Apoptosis. Mol Cell Proteomics 2: 463-473.
5. Missner E, Bahr I, Badock V, Lucking U, Siemeister G, et al. (2009) Off-target decoding of a multitarget kinase inhibitor by chemical proteomics. Chembiochem 10: 1163-1174.
6. Hall S E (2006) Chemoproteomics-driven drug discovery: addressing high attrition rates. Drug Discovery Today 11: 495-502.
7. Alexopoulos L G, Saez-Rodriguez J, Espelin C W (2009) High throughput protein-based technologies and computational models for drug development, efficacy and toxicity. In: Ekins S, Xu J J, editors. pp. 29-52. Drug Efficacy, Safety, and Biologics Discovery: Emerging Technologies and Tools: Wiley.
8. Lamb J, Crawford E D, Peck D, Modell J W, Blat IC, et al. (2006) The Connectivity Map: Using Gene-Expression Signatures to Connect Small Molecules, Genes, and Disease. Science 313: 1929-1935.
9. Iorio F, Tagliaferri R, Bernardo Dd (2009) Identifying Network of Drug Mode of Action by Gene Expression Profiling. Journal of Computational Biology 16: 241-251.
10. di Bernardo D, Thompson M J, Gardner T S, Chobot S E, Eastwood E L, et al. (2005) Chemogenomic profiling on a genome-wide scale using reverse-engineered gene networks. Nat Biotech 23: 377-383.
11. Xing H, Gardner T S (2007) The mode-of-action by network identification (MNI) algorithm: a network biology approach for molecular target identification. Nat Protocols 1: 2551-2554.
12. Szardenings K, Li B, Ma L, Wu M (2004) Fishing for targets: novel approaches using small molecule baits. Drug Discovery Today: Technologies 1: 9-15.
13. Knight Z A, Shokat K M (2005) Features of selective kinase inhibitors. Chem Biol 12: 621-637.
14. Ong S-E, Schenone M, Margolin A A, Li X, Do K, et al. (2009) Identifying the proteins to which small-molecule probes and drugs bind in cells. Proceedings of the National Academy of Sciences 106: 4617-4622.
15. Bantscheff M, Eberhard D, Abraham Y, Bastuck S, Boesche M, et al. (2007) Quantitative chemical proteomics reveals mechanisms of action of clinical ABL kinase inhibitors. Nat Biotech 25: 1035-1044.
16. Daub H, Olsen J V, Bairlein M, Gnad F, Oppermann F S, et al. (2008) Kinase-Selective Enrichment Enables Quantitative Phosphoproteomics of the Kinome across the Cell Cycle. 31: 438-448.
17. L G Alexopoulos*, J Saez-Rodriguez*, B D Cosgrove, D A Lauffenburger, P K Sorger. (*Equal contributors) (2010) Networks inferred from biochemical data reveal profound differences in TLR and inflammatory signaling between normal and transformed hepatocytes. Molecular & Cellular Proteomics doi: 10.1074/mcp.M110.000406.
18. Saez-Rodriguez J, Alexopoulos L G, Epperlein J, Samaga R, Lauffenburger D A, et al. (2009) Discrete logic modelling as a means to link protein signaling networks with functional analysis of mammalian signal transduction Mol Sys Biol. 5:331, 2009.
19. Xia W L, Mullin R J, Keith B R, Liu L H, Ma H, et al. (2002) Anti-tumor activity of GW572016: a dual tyrosine kinase inhibitor blocks EGF activation of EGFR/erbB2 and downstream Erk1/2 and AKT pathways. Oncogene 21: 6255-6263.
20. Norman P (2001) OSI-774 OSI Pharmaceuticals. Curr Opin Investig Drugs 2: 298-304.
21. Baselga J, Averbuch S D (2000) ZD1839 ('Iressa')(1,2) as an anticancer agent. Drugs 60: 33-40.
22. Lee J T, McCubrey J A (2003) BAY-43-9006 Bayer/Onyx. Curr Opin Investig Drugs 4: 757-763.
23. Nagasaki M, Saito A, Doi A, Matsuno H, Miyano S (2009) Pathway Databases. Foundations of Systems Biology 5-18.
24. Jensen L J, Saric J, Bork P (2006) Literature mining for the biologist: from information retrieval to biological discovery. Nature Reviews Genetics 7: 119-129.
25. Samaga R, Saez-Rodriguez J, Alexopoulos L G, Sorger P K, Klamt S (2009) The Logic of EGFR/ErbB Signaling: Theoretical Properties and Analysis of High-Throughput Data. PLoS Comput Biol accepted.
26. Gupta S, Bisht S S, Kukreti R, Jain S, Brahmachari S K (2007) Boolean network analysis of a neurotransmitter signaling pathway. Journal of Theoretical Biology 244: 463-469.
27. Klamt S, Saez-Rodriguez J, Gilles E (2007) Structural and functional analysis of cellular networks with CellNetAnalyzer. BMC Systems Biology 1:2.
28. Thomas R, D'Ari R (1990) Biological feedback. Boca Raton, Fla., USA: CRC Press.
29. Klamt S, Haus U-U, Theis F (2009) Hypergraphs and Cellular Networks. PLoS Comput Biol 5: e1000385.
30. ILOG SA (2003) ILOG CPLEX 9.0 Reference Manual, www.ilog.com/products/cplex/.
31. Brooke A, Kendrick D, Meeraus A (1988) GAMS: User's Guide. Redwood City, Calif.: The Scientific Press.
32. Karaman M W, Herrgard S, Treiber D K, Gallant P, Atteridge C E, et al. (2008) A quantitative analysis of kinase inhibitor selectivity. Nat Biotech 26: 127-132.
33. Wilhelm S M, Carter C, Tang L, Wilkie D, McNabola A, et al. (2004) BAY 43-9006 Exhibits Broad Spectrum Oral Antitumor Activity and Targets the RAF/MEK/ERK Pathway and Receptor Tyrosine Kinases Involved in Tumor Progression and Angiogenesis. Cancer Res 64: 7099-7109.
34. Rual J-F, Venkatesan K, Hao T, Hirozane-Kishikawa T, Dricot A, et al. (2005) Towards a proteome-scale map of the human protein-protein interaction network. Nature 437: 1173-1178.
35. Sachs K, Perez O, Pe'er D, Lauffenburger D A, Nolan G P (2005) Causal Protein-Signaling Networks Derived from Multiparameter Single-Cell Data. Science 308: 523-529.
36. Kocher T, Superti-Furga G (2007) Mass spectrometry-based functional proteomics: from molecular machines to protein networks. Nat Meth 4: 807-815.
37. MacBeath G, Schreiber S L (2000) Printing Proteins as Microarrays for High-Throughput Function Determination. Science 289: 1760-1763.
38. Jansen R, Yu H, Greenbaum D, Kluger Y, Krogan N J, et al. (2003) A Bayesian Networks Approach for Predicting Protein-Protein Interactions from Genomic Data. Science 302: 449-453.
39. Gaudet S, Janes K A, Albeck J G, Pace E A, Lauffenburger D A, et al. (2005) A compendium of signals and responses triggered by prodeath and prosurvival cytokines. Mol Cell Proteomics M500158-MCP500200.
40. Janes K A, Kelly J R, Gaudet S, Albeck J G, Sorger P K, et al. (2004) Cue-signal-response analysis of TNF-induced apoptosis by partial least squares regression of dynamic multivariate data. J Comput Biol 11: 544-561.
41. Nelander S, Wang W, Nilsson B, She Q-B, Pratilas C, et al. (2008) Models from experiments: combinatorial drug perturbations of cancer cells. Mol Syst Biol 4:216.
42. Kauffman SA (1969) Metabolic stability and epigenesis in randomly constructed genetic nets. J Theor Biol 22: 437-467.
43. Aldridge B B, Saez-Rodriguez J, Muhlich J L, Sorger P K, Lauffenburger D A (2009) Fuzzy Logic Analysis of Kinase Pathway Crosstalk in TNF/EGF/Insulin-Induced Signaling. PLoS Comput Biol 5: e1000340.
44. Mendoza L, Xenarios I (2006) A method for the generation of standardized qualitative dynamical systems of regulatory networks. Theor Biol Med Model 3: 13.
45. Chaparro M, Gonzalez M L, M T-M, J M, R M-O (2008) Review article: pharmacological therapy for hepatocellular carcinoma with sorafenib and other oral agents. Alimentary Pharmacology & Therapeutics 28: 1269-1277.
46. Schoeberl B, Pace E A, Fitzgerald J B, Harms B D, Xu L, et al. (2009) Therapeutically Targeting ErbB3: A Key Node in Ligand-Induced Activation of the ErbB Receptor-PI3K Axis. Sci Signal 2: ra31.
47. Wood E R, Truesdale A T, McDonald O B, Yuan D, Hassell A, et al. (2004) A Unique Structure for Epidermal Growth Factor Receptor Bound to GW572016 (Lapatinib): Relationships among Protein Conformation, Inhibitor Off-Rate, and Receptor Activity in Tumor Cells. Cancer Res 64: 6652-6659.
48. Saez-Rodriguez J, Goldsipe A, Muhlich J, Alexopoulos L G, Millard B, et al. (2008) Flexible informatics for linking experimental data to mathematical models via DataRail. Bioinformatics 24: 840-847.
49. Haus U U, Niermann K, Truemper K, Weismantel R (2009) Logic integer programming models for signaling networks. J Comput Biol 16: 725-743.
50. Clark P A, Westerberg A W (1983) Optimization for Design Problems having more than one objective. Computers & Chemical Engineering 7: 259-278.
51. Ahmad B S, Barton P I (1999) Process-wide integration of solvent mixtures. Computers & Chemical Engineering 23: 1365-1380.
52. Selot A, Kuok L K, Robinson M, Mason T L, Barton PI (2008) A short-term operational planning model for natural gas production systems. AIChE Journal 54: 495-515.
53. Mitsos A, Oxberry G M, Barton P I, Green W H (2008) Optimal automatic reaction and species elimination in kinetic mechanisms. Combustion and Flame 155: 118-132.
54. Shannon P, Markiel A, Ozier O, Baliga N S, Wang J T, et al. (2003) Cytoscape: A Software Environment for Integrated Models of Biomolecular Interaction Networks. Genome Research 13: 2498-2504.

All publications, patents, database, and database entries mentioned herein, including those items listed below, are hereby incorporated by reference in their entirety as if each individual publication, patent, database, or database entry was specifically and individually indicated to be incorporated by reference. In case of conflict, the present application, including any definitions herein, will control.

### EQUIVALENTS AND SCOPE

Those skilled in the art will recognize, or be able to ascertain using no more than routine experimentation, many equivalents to the specific embodiments of the invention described herein. The scope of the present invention is not intended to be limited to the above description, but rather is as set forth in the appended claims.

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or all of the group members are present in, employed in, or otherwise relevant to a given method, formulation, or process.

Furthermore, it is to be understood that the invention encompasses all variations, combinations, and permutations in which one or more limitations, elements, clauses, descriptive terms, formulations, constraints, etc., from one or more of the claims or from relevant portions of the description is introduced into another claim. For example, any claim that is dependent on another claim can be modified to include one or more limitations found in any other claim that is dependent on the same base claim.

Where elements are presented as lists, e.g., in Markush group format, or as lists within the specification, it is to be understood that each subgroup of the elements is also disclosed, and any element(s), formulation, constraint, etc., can be removed from the group. It is also noted that the term “comprising” is intended to be open and permits the inclusion of additional elements or steps. It should be understood that, in general, where the invention, or aspects of the invention, is/are referred to as comprising particular elements, features, steps, formulations, constraints, etc., certain embodiments of the invention or aspects of the invention consist, or consist essentially of, such elements, features, steps, formulations, constraints, etc. For purposes of simplicity those embodiments have not been specifically set forth in haec verba herein. Thus for each embodiment of the invention that comprises one or more elements, features, steps, formulations, constraints, etc., the invention also provides embodiments that consist or consist essentially of those elements, features, steps, formulations, constraints, etc.

Where ranges are given, endpoints are included. Furthermore, it is to be understood that unless otherwise indicated or otherwise evident from the context and/or the understanding of one of ordinary skill in the art, values that are expressed as ranges can assume any specific value within the stated ranges in different embodiments of the invention, to the tenth of the unit of the lower limit of the range, unless the context clearly dictates otherwise. It is also to be understood that unless otherwise indicated or otherwise evident from the context and/or the understanding of one of ordinary skill in the art, values expressed as ranges can assume any subrange within the given range, wherein the endpoints of the subrange are expressed to the same degree of accuracy as the tenth of the unit of the lower limit of the range. Further, it is to be understood that any value or subrange within a range can be excluded from the scope of this invention.

In addition, it is to be understood that any particular embodiment of the present invention may be explicitly excluded from any one or more of the claims. Where ranges are given, any value within the range may explicitly be excluded from any one or more of the claims. Any embodiment, element, feature, application, or aspect of the compositions and/or methods of the invention, can be excluded from any one or more claims. For purposes of brevity, all of the embodiments in which one or more elements, features, purposes, or aspects is excluded are not set forth explicitly herein.

1. A method of generating a cell-specific signaling topology using integer linear programming, the method comprising
(a) providing a generic cellular signaling topology comprising a signaling pathway, wherein the signaling pathway comprises a plurality of nodes representing signaling molecules, and wherein the nodes are connected via edges representing reactions within the pathway;
(b) determining a quantitative baseline value for a plurality of designated nodes in the generic signaling topology by measuring a quantitative value for each of the plurality of designated nodes in a cell;
(c) determining a quantitative value for each of the plurality of designated nodes in the generic signaling topology by measuring a quantitative value for each of the plurality of designated nodes in a cell of the same type as the cell in (b) contacted with a stimulus known to perturb the generic signaling topology;
optionally, wherein step (c) comprises a plurality of separate measurements of cells contacted with different stimuli known to perturb the generic signaling topology;
(d) comparing the quantitative baseline value for each of the plurality of designated nodes with the quantitative value for that node determined in the presence of the stimulus known to perturb the generic signaling topology;
(e) generating a cell-specific signaling topology by modifying the generic signaling topology and using an integer linear programming (ILP) algorithm to identify the modified signaling topology that best fits the values determined in (b) and (c) as the cell-specific signaling topology;
optionally, wherein step (e) comprises
(e.1) determining the fit of the generic model to the observed data;
(e.2) modifying the generic signaling topology by adding and/or removing reactions to generate a plurality of modified signal topologies;
(e.3) determining the fit of each or of a subset of the plurality of modified signal topologies to the observed data; and
(e.4) selecting the modified signal topology exhibiting the best fit to the experimental data as the cell-specific signal topology.

2. The method of claim 1, wherein the ILP algorithm defines a pathway as a set of reactions and species, wherein each species is represented by a node in the pathway topology and each reaction is represented by an edge in the pathway topology.

3. The method of claim 2, wherein a reaction is defined by three index sets: (1) a set of signaling molecules, (2) a set of perturbations, and (3) a set of reaction products.

4-7. (canceled)

8. The method of claim 1, wherein the ILP algorithm comprises
a constraint defining that a reaction takes place only if all reagents and no inhibitors are present;
a constraint defining that if a reaction takes place, all products are formed; and/or
a constraint excluding reactions without products and/or reactions with neither reagents nor inhibitors.

9-10. (canceled)

11. The method of claim 1,
wherein the ILP algorithm comprises integer or binary decision variables indicating if a reaction is possible or not;
optionally, wherein at least one of the ILP decision variables is relaxed to continuous, wherein,
optionally, wherein the relaxation of the at least one of the ILP decision variables does not alter the feasible set; and
optionally, wherein at least one of the ILP decision variables is a real decision variable.

12-20. (canceled)

21. The method of claim 1, wherein the modified topology or group of modified topologies with the best fit is/are selected as the cell-specific topology or group of topologies.

22. The method of claim 1, further comprising
(f) optimizing the cell-specific topology to minimize the number of nodes and/or possible reactions without worsening the fit of the topology to the experimental data.

23. The method of claim 1,
wherein the ILP algorithm is the ILP algorithm as provided in formulation (1), or a reformulation thereof;
wherein the ILP algorithm comprises a constraint as provided in any of formulations (2)-(11), or a reformulation thereof; or
wherein the ILP algorithm is the ILP algorithm as provided in formulations (1)-(20), or a reformulation thereof.

24-35. (canceled)

35. A method for identifying an effect of a drug on a molecular signaling pathway in a cell using integer linear programming, the method comprising
(a) providing a cell-specific signaling topology comprising a signaling pathway, wherein the signaling pathway comprises nodes representing signaling molecules, and wherein the nodes are connected via edges representing reactions within the pathway, and wherein the cell-specific signaling topology is based on quantitative values measured in a cell for a plurality of designated nodes comprised in the signaling topology in the absence and the presence of a stimulus known to perturb the signaling pathway,
(b) determining drug-induced signaling topology alterations by
(i) contacting a cell of the same cell type as the cell in (a) with a drug known or suspected to perturb at least one reaction in the topology provided in (a), in the absence and/or in the presence of the stimulus known to perturb the signaling pathway;
(ii) measuring a quantitative value for a plurality of designated nodes in the cell-specific signaling topology in a cell contacted with the drug in the absence and/or the presence of the stimulus known to perturb the cell-specific signaling topology,
(iii) generating a cell-specific, drug-induced signaling topology by using an ILP algorithm to adapt the cell-specific signaling topology to best fit the values measured in (b)(ii);
optionally, wherein step (b)(iii) comprises
(b.iii.1) determining the fit of the cell-specific signaling topology to the data observed in the presence of the drug;
(b.iii.2) modifying the cell-specific signaling topology by adding and/or removing reactions to generate a plurality of modified signal topologies.,
(b.iii.3) determining the fit of each of the plurality of modified signal topology to the data observed in the presence of the drug; and
(b.iii.4) selecting the modified signal topology exhibiting the best fit to the experimental data as the cell-specific, drug-induced signal topology; and
(c) comparing the cell-specific signaling topology provided in (a) to the cell-specific, drug-induced signaling topology generated in (b)(iii), wherein
(i) if a reaction is present in the cell-specific signaling topology but absent in the cell-specific, drug-induced signaling topology, the reaction is indicated to be inhibited by the drug;
(ii) if a reaction is absent in the cell-specific signaling topology but present in the cell-specific, drug-induced signaling topology, the reaction is indicated to be activated by the drug; and/or
(iii) if a reaction is either absent or present in both the cell-specific and the cell-specific, drug-induced signaling topology, the reaction is indicated to not be affected by the drug.

36. The method of claim 35, wherein the ILP algorithm defines a pathway as a set of reactions and species, wherein each species is represented by a node in the pathway topology and each reaction is represented by an edge in the pathway topology.

37. The method of claim 36, wherein a reaction is defined by three index sets: (1) a set of signaling molecules, (2) a set of perturbations, and (3) a set of reaction products.

38-41. (canceled)

42. The method of claim 35, wherein the ILP algorithm comprises
a constraint defining that a reaction takes place only if all reagents and no inhibitors are present;
a constraint defining that if a reaction takes place, all products are formed; and/or
constraint excluding reactions without products and/or reactions with neither reagents nor inhibitors.

43-44. (canceled)

45. The method of claim 35, wherein the ILP algorithm comprises integer or binary decision variables indicating if a reaction is possible or not;
optionally, wherein at least one of the ILP decision variables is relaxed to continuous;
optionally, wherein the relaxation of the at least one of the ILP decision variables does not alter the feasible set; and
optionally, wherein at least one of the ILP decision variables is a real decision variable.

46-55. (canceled)

55. The method of claim 35, wherein the modified topology with the lowest percentage error is selected as the cell-specific topology.

56. The method of claim 35,
wherein the ILP algorithm is the ILP algorithm as provided in formulation (1), or a reformulation thereof;
wherein the ILP algorithm comprises a constraint as provided in any of formulations (2)-(4) and/or (6)-(11), or a reformulation thereof
wherein the ILP algorithm comprises a constraint as provided in formulation (5), or a reformulation thereof;
wherein the ILP algorithm is the ILP algorithm as provided in formulations (1)-(4) and (6)-(11), or a reformulation thereof; or
wherein the ILP algorithm is the ILP algorithm as provided in formulations (1)-(11), or a reformulation thereof.

57-69. (canceled)

70. A method for selecting and/or administering a drug targeting a signaling molecule or reaction for treatment of a subject diagnosed with a disease based on a signaling pathway topology assessment, the method comprising
(a) generating or obtaining a cell-specific signaling topology from a cell population obtained from a subject, wherein the cell population comprises cells in a diseased state and wherein the signaling topology is generated by using an integer linear programming optimization scheme;
(b) comparing the cell-specific signaling topology from the subject to a reference signaling topology; wherein
(i) if a signaling molecule and/or reaction is absent in the reference topology and present in the topology of the subject, then treatment of the subject with a drug that inhibits the signaling molecule and/or reaction is indicated; or
(ii) if a signaling molecule and/or reaction is present in the reference topology and absent in the topology of the subject, then treatment of the subject with a drug that activates the signaling molecule and/or reaction is indicated.

71. The method of claim 70, further comprising
(c) administering the drug indicated under (i) or (ii) to the subject in an amount sufficient to inhibit or activate the signaling molecule and/or reaction.

72. The method of claim 70, wherein the cell population is derived from a diseased tissue of the subject.

73-76. (canceled)

77. The method of claim 70, wherein the drug is chosen from a group of EGFR inhibitors.

78. The method of claim 77, wherein the group of EGFR inhibitors comprises Lapatinib, Gefitinib, and Erlotinib.

79. The method of claim 70, wherein the reference topology is derived or representative of non-diseased cells of the same cell type or tissue of origin as the cells obtained from the subject.

80. A method for selecting and/or administering a drug targeting a signaling molecule or reaction for treatment of a subject diagnosed with a disease based on a signaling pathway topology assessment, the method comprising
(a) generating or obtaining a cell-specific signaling topology from a cell population obtained from a subject, wherein the cell population comprises cells in a diseased state;
(b) determining whether a reaction or signaling molecule known to be targeted by a particular drug is present or absent in the cell-specific signaling topology, and
(c) if the reaction or signaling molecule known to be targeted by the particular drug is absent, administering the drug to the subject in an amount sufficient to modulate the reaction or signaling molecule to achieve a desired clinical outcome, or
if the reaction or signaling molecule known to be targeted by the particular drug is absent, not administering the drug to the subject.

81. The method of claim 80, wherein the cell population is derived from a diseased tissue of the subject.

82-85. (canceled)

86. The method of claim 80, wherein the drug is chosen from a group of EGFR inhibitors.

87. The method of claim 86, wherein the group of EGFR inhibitors comprises Lapatinib, Gefitinib, and Erlotinib.
